SEQUENCE LISTING (10 sequences)

SEQ ID NO: 1 - DNA, 8835 bp, Homo sapiens
atgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagcccaccagtgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccagctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacagggcctccctggcccctctggtccccctggattaccgggaaagccaggcgaggatggcaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactga

SEQ ID NO: 2 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 3 - DNA, 4494 bp, Homo sapiens
atggatgtaaccaagaaaaacaaacgagatggaactgaagtcactgagagaattgtcactgaaacagtaaccacaagacttacatccttaccaccaaaaggcgggaccagcaatggctatgctaaaacagcctctcttggtggagggagccggctggagaaacaaagcctgactcatggcagcagcggctacataaactcaactggaagcacacgaggccatgcctccacctctagttacaggagggctcactcacctgcctccactctgcccaactccccaggctcaacctttgaaaggaaaactcacgttacccgccatgcgtatgaagggagctccagtggcaactcttctccggagtaccctcggaaggaatttgcatcttcttcaaccagaggacggagtcaaacacgagagagtgaaattcgagttcgactgcagagtgcgtccccatccacccgatggacagaattggatgatgttaagcgtttgctcaaggggagtcgatcggcaagtgtgagccccacccggaattcctccaacacactccccatccccaagaaaggcactgtggagaccaaaattgtgacagcgagctcccagtcggtgtcaggcacctacgatgcaacgatcctggatgccaaccttccctcccatgtgtggtcctccaccctgcccgcggggtcctccatggggacctatcacaacaacatgacaacccagagctcatccctcctcaacaccaatgcctactctgcgggatcagtcttcggagttccaaacaacatggcgtcctgctcacccactttgcaccctggactcagcacatcctcctcagtgtttggcatgcagaacaatctggcccccagcttgaccaccctgtcccatggcaccaccaccacttccacagcatatggggtgaagaaaaacatgccccagagtcctgcggctgtgaacactggcgtttccacctccgccgcctgcaccacaagtgtgcagagcgatgaccttttgcacaaggactgcaagttcctgatcctagagaaagacaacacacctgccaagaaggagatggagctgctcatcatgaccaaggacagcgggaaggtctttacagcctcccctgccagcatcgctgcaacttctttttcagaagacaccctaaaaaaagaaaagcaagctgcctacaatgctgactcaggcctaaaagccgaagctaatggagacctgaagactgtgtccacaaagggcaagaccaccactgcagatatccacagctacggcagcagtggtggtggtggcagtggaggaggtggcggtgttggtggcgctggcggcggcccttggggaccagcgccagcctggtgcccctgcggctcctgctgcagctggtggaagtggctgctgggcctgctgctcacctggctgctactcctggggctgctcttcggcctcattgctctggcggaggaggtgaggaagctgaaggcgcgtgtggatgagctggagaggatcaggaggagcatactgccctatggggacagcatggatagaatagaaaaggaccgcctccagggcatggcacccgcggcgggagcagacctggacaaaattgggctgcacagtgacagccaggaggagctctggatgttcgtgaggaagaagctaatgatggaacaggaaaatggaaatctccgaggaagccctggccctaaaggtgacatgggaagtccaggccctaaaggagatcgagggttccctgggactccaggtatccctgggcccttgggccacccaggtccacaaggaccaaagggtcaaaaaggcagcgtgggagatcctggcatggaaggccccatgggccagagagggcgagaaggccccatgggacctcgtggtgaggcagggcctcctggatctggagagaaaggggaaagaggggctgctggtgaaccaggtcctcatggcccacctggtgtcccaggttctgtgggtcccaaaggttccagcggctctcctggcccacagggccctccaggtcctgtaggtctccaagggctccgaggtgaagtaggacttcctggtgtcaaaggtgacaaaggaccaatgggaccaccaggacccaaaggtgaccagggtgagaaaggacctcgaggcctcacaggcgagcctggcatgagaggtttgcctggtgctgttggtgagcccggggctaaaggagcaatgggtcctgctggcccagacggacaccaaggcccaagaggtgaacaaggtcttactgggatgcctggaatccgtggcccaccaggaccttctggagacccaggaaagccaggtctcacaggaccccagggacctcagggacttcccggtacccctggccgaccaggaataaaaggtgaaccaggagctccaggcaagatcgtgacttcggaggggtcatcgatgctcactgtcccaggccccccaggacctcctggagccatgggacccccaggacctccaggtgccccaggccctgccggcccagctggtctcccaggacatcaagaagttcttaatttacaaggtcccccaggcccacccggcccacgcgggccaccagggccttccattccaggcccaccaggaccccgaggcccaccaggggagggtttgccaggcccaccaggcccaccaggatcgttcctgtccaactcagaaaccttcctctccggccccccaggcccacctggccccccaggtcccaagggagaccaaggtcccccaggccccagaggacaccaaggcgagcaaggcctcccaggtttctcaacctcagggtccagttctttcggactcaaccttcagggaccaccaggcccacctggcccccagggacccaaaggtgacaaaggtgatccaggtgttccaggggctcttggcattcctagtggtccttctgaagggggatcatcaagtaccatgtacgtgtcaggcccgccagggccccctgggccccctgggcctccgggctctatcagcagctctggccaggagattcagcagtacatctctgagtacatgcagagtgacagtattagatcttacctatccggagttcagggtcccccaggcccacctggtcccccaggacctgtcaccaccatcacaggcgagactttcgactactcagagctggcaagccacgttgtgagctacttacggacttcggggtacggtgtcagcttgttctcgtcctccatctcttctgaagacattctggctgtgctgcagcgggatgacgtgcgtcagtacctacgtcagtacttgatgggccctcggggtccgccagggccaccaggagccagtggagatgggtccctcctgtctttggactatgcagagctgagtagtcgcattctcagctacatgtcgagttctgggatcagcattgggcttcctggtcccccggggccccctggcttgccgggaacctcctatgaggagctcctctccttgctgcgagggtctgaattcagaggcatcgttggacccccaggtcccccgggtccaccagggatcccaggcaatgtgtggtccagcatcagcgtggaggacctctcgtcttacttacatactgccggcttgtcattcatcccaggccctccaggacctcctggtcccccagggcctcgagggcccccgggtgtctcaggagccctggcaacctatgcagctgaaaacagcgacagcttccggagcgagctgatcagctacctcacaagtcctgatgtgcgcagcttcattgttggccccccaggccctcctgggccgcagggaccccctggggacagccgcctcctgtccacggatgcctcccacagtcggggtagcagctcctcctcacacagctcatctgtcaggcggggcagctcctacagctcttccatgagcacaggaggaggtggtgcaggctccctgggtgcaggcggtgcctttggtgaagctgcaggagacaggggtccctatggcactgacatcggcccaggcggaggctatggggcagcagcagaaggcggcatgtatgctggcaatggcggactattgggagctgactttgctggagatctggattacaatgagctggctgtgagggtgtcagagagcatgcagcgtcagggcctactgcaagggatggcctacactgtccagggcccaccaggccagcctgggccacaggggccacccggcatcagcaaggtcttctctgcctacagcaacgtgactgcggacctcatggacttcttccaaacttatggagccattcaaggaccccctgggcaaaaaggagagatgggcactccaggacccaaaggtgacaggggccctgctgggccaccaggtcatcctgggccacctggccctcgaggacacaagggagaaaaaggagacaaaggtgaccaagtctatgctgggcggagaaggagaagaagtattgctgtcaagccgtga

SEQ ID NO: 4 - protein, 1497 aa, Homo sapiens
MDVTKKNKRDGTEVTERIVTETVTTRLTSLPPKGGTSNGYAKTASLGGGSRLEKQSLTHGSSGYINSTGSTRGHASTSSYRRAHSPASTLPNSPGSTFERKTHVTRHAYEGSSSGNSSPEYPRKEFASSSTRGRSQTRESEIRVRLQSASPSTRWTELDDVKRLLKGSRSASVSPTRNSSNTLPIPKKGTVETKIVTASSQSVSGTYDATILDANLPSHVWSSTLPAGSSMGTYHNNMTTQSSSLLNTNAYSAGSVFGVPNNMASCSPTLHPGLSTSSSVFGMQNNLAPSLTTLSHGTTTTSTAYGVKKNMPQSPAAVNTGVSTSAACTTSVQSDDLLHKDCKFLILEKDNTPAKKEMELLIMTKDSGKVFTASPASIAATSFSEDTLKKEKQAAYNADSGLKAEANGDLKTVSTKGKTTTADIHSYGSSGGGGSGGGGGVGGAGGGPWGPAPAWCPCGSCCSWWKWLLGLLLTWLLLLGLLFGLIALAEEVRKLKARVDELERIRRSILPYGDSMDRIEKDRLQGMAPAAGADLDKIGLHSDSQEELWMFVRKKLMMEQENGNLRGSPGPKGDMGSPGPKGDRGFPGTPGIPGPLGHPGPQGPKGQKGSVGDPGMEGPMGQRGREGPMGPRGEAGPPGSGEKGERGAAGEPGPHGPPGVPGSVGPKGSSGSPGPQGPPGPVGLQGLRGEVGLPGVKGDKGPMGPPGPKGDQGEKGPRGLTGEPGMRGLPGAVGEPGAKGAMGPAGPDGHQGPRGEQGLTGMPGIRGPPGPSGDPGKPGLTGPQGPQGLPGTPGRPGIKGEPGAPGKIVTSEGSSMLTVPGPPGPPGAMGPPGPPGAPGPAGPAGLPGHQEVLNLQGPPGPPGPRGPPGPSIPGPPGPRGPPGEGLPGPPGPPGSFLSNSETFLSGPPGPPGPPGPKGDQGPPGPRGHQGEQGLPGFSTSGSSSFGLNLQGPPGPPGPQGPKGDKGDPGVPGALGIPSGPSEGGSSSTMYVSGPPGPPGPPGPPGSISSSGQEIQQYISEYMQSDSIRSYLSGVQGPPGPPGPPGPVTTITGETFDYSELASHVVSYLRTSGYGVSLFSSSISSEDILAVLQRDDVRQYLRQYLMGPRGPPGPPGASGDGSLLSLDYAELSSRILSYMSSSGISIGLPGPPGPPGLPGTSYEELLSLLRGSEFRGIVGPPGPPGPPGIPGNVWSSISVEDLSSYLHTAGLSFIPGPPGPPGPPGPRGPPGVSGALATYAAENSDSFRSELISYLTSPDVRSFIVGPPGPPGPQGPPGDSRLLSTDASHSRGSSSSSHSSSVRRGSSYSSSMSTGGGGAGSLGAGGAFGEAAGDRGPYGTDIGPGGGYGAAAEGGMYAGNGGLLGADFAGDLDYNELAVRVSESMQRQGLLQGMAYTVQGPPGQPGPQGPPGISKVFSAYSNVTADLMDFFQTYGAIQGPPGQKGEMGTPGPKGDRGPAGPPGHPGPPGPRGHKGEKGDKGDQVYAGRRRRRSIAVKP

SEQ ID NO: 5 - DNA, 3519 bp, Homo sapiens
atgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcacagaatgatgtgaaccctgtctctctgcagctggacctggacaggagattccagcttcaagaagtcatgatggagttccaggggcccatgcccgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactccctcagcccacagtgcaaccagttcacagggcagtgcccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgtaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtga

SEQ ID NO: 6 - protein, 1172 aa, Homo sapiens [sequence omitted]

SEQ ID NO: 7 - DNA, 9834 bp, Homo sapiens
atggcggcggccgcgcggcctcggggtcgggcactggggccagtactgccgccgacgccgctgctcctgctggtactgcgggtgctgccagcctgcggggcgaccgctcgggatcccggggccgcggccgggctcagccttcacccgacttacttcaacctggccgaggcggcgaggatttgggccaccgccacctgcggggagaggggacccggcgaggggaggccccagcccgagctctactgcaagttggtcgggggccccaccgccccaggcagcggccacaccatccagggccagttctgtgactattgcaattctgaagaccccaggaaagcacatcctgtcaccaatgccatcgatggatctgaacgttggtggcaaagccctcccctgtcctcaggcacacagtacaacagagtcaacctcaccttggatctggggcagctcttccatgtggcctatattttaatcaaatttgcaaattctcctcgccctgatctttgggtcttggaaagatctgtagactttggaagcacctactcaccatggcaatattttgctcattctaaagtagactgtttaaaagaatttgggcgggaggcaaatatggctgtcacccgggatgatgatgtactttgtgttactgaatattcccgtattgtacctttggaaaatggtgaggttgtggtgtccttgataaacggtcgtccaggtgcaaaaaattttactttctctcacaccctgagggagtttaccaaggcaacaaacatccgcttgcgttttcttagaaccaatacgcttcttggacacctcatctccaaagcccagcgagatccaactgtcactcggcggtattattacagcataaaggacatcagcattggtgggcagtgtgtttgcaatggccatgctgaagtgtgcaatataaacaatcctgaaaaactgtttcggtgtgaatgccagcaccacacctgtggggagacgtgtgatcgctgctgcacagggtacaatcagaggcgctggcggcccgccgcttgggagcagagccacgagtgtgaagcatgcaactgccacggccatgccagcaactgttactatgatccagatgttgagcggcagcaggcaagcttgaatacccagggcatctatgctggtggaggggtctgcattaactgtcagcacaacacagctggagtaaactgtgaacagtgtgctaagggctattaccgcccttatggggttccagtggatgcccctgatggctgcatcccctgcagctgtgaccctgagcatgcggatggctgtgaacagggttcaggccgctgtcactgcaagccaaatttccacggagacaactgtgagaagtgtgcaattggatactacaatttcccattttgcttgagaattcccatttttcctgtttctacaccaagttcagaagatccagtagctggagatataaaagggtgtgactgtaatctggaaggtgttctccctgaaatatgtgatgcccacggacggtgcctgtgccgccctggggttgagggccctcgatgtgatacctgccgctctggtttctactcattccctatttgccaagcctgctggtgttcagcccttggatcctaccagatgccctgcagctcagtgactggacagtgtgaatgtcggccaggagttacaggacagcggtgtgacaggtgtctctcaggagcttatgatttcccccactgccaaggttccagcagtgcttgtgacccagctggtaccatcaactccaatttggggtattgccaatgcaagcttcatgttgaaggtcctacttgtagccgctgcaaactgttatattggaatctggacaaagaaaaccccagtggatgttcagaatgcaagtgccataaggcgggaacagtgagtggaactggagagtgtaggcagggagatggtgactgtcactgcaagtcccatgtgggtggcgattcctgcgacacctgtgaagatggatattttgctttggaaaagagcaattactttgggtgtcaagggtgtcagtgtgacattggtggggcattgtcctccatgtgcagtgggccctcgggagtgtgccagtgccgagagcatgtcgtgggaaaggtgtgccagcggcctgaaaacaactactatttcccagatttgcatcatatgaagtatgagattgaagacggcagcacacctaatgggagagaccttcgatttggatttgatccgctggcatttcctgagtttagctggagaggatatgcccaaatgacctcagtacagaatgatgtaagaataacattgaatgtagggaagtcaagtggctccttgtttcgtgttattctgagatacgttaaccctggaactgaagcagtatctggccatataactatttatccatcctggggtgctgctcaaagcaaagagatcatcttcctgccgagtaaggagccagcctttgtcactgtccctggaaatggttttgcagacccattttcaatcacaccaggaatatgggttgcttgtattaaggcagaaggagtccttctggattacctggtgctgctccccagggactactatgaagcctctgtactgcagctgccagtcacagaaccatgtgcctacgcaggacctccccaagaaaattgcttactctaccagcatttgccagtgaccagattcccctgtaccctggcttgtgaggccagacacttcctgcttgatggggagccaagacccgtggcagtgaggcagcccacacctgcacaccctgtcatggtggacctcagcgggagagaggtggaattgcatctgcggctgcgcatcccacaggttggccactacgtggttgtggtcgagtattccacggaggcagctcagctgtttgtggttgatgtgaatgtgaagagctccgggtctgttctggcaggccaggtgaacatttacagctgcaactacagtgttctctgccggagtgctgtgattgatcacatgagccgcatcgccatgtatgagctattggcagatgcagacattcagctcaagggacacatggcccgattccttctgcatcaagtttgtatcatacctattgaagaattctcagctgagtatgtgagaccacaagtccactgcattgccagttatgggcgatttgtcaatcaaagtgccacctgtgtctccttggcccatgaaactcctccaacagcattaattttggatgttctaagtggcaggcctttccctcacctgccccagcagtcgtcaccttctgttgatgttcttcctggggtcaccttgaaggcaccgcagaatcaagtgaccctgagaggacgtgtaccacacctgggccgatacgtctttgtcatccatttttaccaagcagcgcacccgacgtttcccgcgcaggtgtcggtggatggcgggtggccacgggcaggctccttccatgcctctttttgcccccatgtgcttggctgccgggatcaagtgattgccgaaggccagattgagtttgacatctcagagcctgaagtggccgcaactgtgaaggttccagaaggaaagtccttggttttggtccgtgttctagtggtgcctgcagaaaactatgactaccaaatacttcacaaaaaatccatggacaagtcactcgagtttatcaccaattgtggaaaaaacagcttttaccttgacccccagacagcctccagattctgtaagaattccgccaggtccctggtggccttttaccacaagggcgccctgccttgtgagtgccaccccactggggccaccggccctcactgcagccctgagggtgggcagtgcccatgccagcccaacgtcatcgggcggcagtgcacccgctgtgcaacaggccactacggattcccacgctgcaagccgtgcagctgtggtcggcgcctttgtgaagagatgacggggcagtgccgctgccctccccgcacggtcaggccccagtgtgaggtgtgtgagacacactcattcagcttccaccccatggccggctgcgaaggctgcaactgttccaggaggggcaccatcgaggctgccatgccggagtgtgaccgggacagcgggcagtgcagatgcaagcccagaatcacagggcggcagtgtgaccgatgtgcttccgggttttaccgctttcctgagtgtgttccctgcaattgcaacagagatgggactgagccaggagtgtgtgacccagggaccggggcttgcctctgcaaggaaaatgtagaaggcacagagtgtaatgtgtgtcgagaaggctcattccatttggacccagccaatctcaagggttgtaccagctgtttctgttttggagtaaataatcaatgtcacagctcacataagcgaaggactaagtttgtggatatgctgggctggcacctggagacagcagacagagtggacatccctgtctctttcaacccaggcagcaacagtatggtggcggatctccaggagctgcccgcaaccatccacagcgcgtcctgggtcgcacccacctcctacctgggggacaaggtttcttcatatggtggttacctcacttaccaagccaagtcctttggcttgcctggcgacatggttcttctggaaaagaagccggatgtacagctcactggtcagcacatgtccatcatctatgaggagacaaacaccccacggccagaccggctgcatcatggacgagtgcacgtggtcgagggaaacttcagacatgccagcagccgtgccccagtgtctagggaggagctgatgacagtgctgtctagactggcagatgtgcgcatccaaggcctctacttcacagagactcaaaggctcaccctgagcgaggtggggctagaggaagcctctgacacaggaagtgggcgcatagcacttgctgtggaaatctgtgcctgcccccctgcctacgctggtgactcttgtcagggttgtagccctggatactatcgggatcataaaggcttgtataccggacggtgtgttccctgcaattgcaacggacattcaaatcaatgccaggatggctcaggcatatgtgttaactgtcagcacaacaccgcgggagagcactgtgaacgctgccaggagggctactatggcaacgccgtccacggatcctgcagggcctgcccatgtcctcacactaacagctttgccactggctgtgtggtgaatgggggagacgtgcggtgctcctgcaaagctgggtacacaggaacacagtgtgaaaggtgtgcaccgggatatttcgggaatccccagaaattcggaggtagctgccaaccatgcagttgtaacagcaatggccagctgggcagctgtcatcccctgactggagactgcataaaccaagaacccaaagatagcagccctgcagaagaatgtgatgattgcgacagctgtgtgatgaccctcctgaacgacctggccaccatgggcgagcagctccgcctggtcaagtctcagctgcagggcctgagtgccagcgcagggcttctggagcagatgaggcacatggagacccaggccaaggacctgaggaatcagttgctcaactaccgttctgccatttcaaatcatggatcaaaaatagaaggcctggaaagagaactgactgatttgaatcaagaatttgagactttgcaagaaaaggctcaagtaaattccagaaaagcacaaacattaaacaacaatgttaatcgggcaacacaaagcgcaaaagaactggatgtgaagattaaaaatgtcatccggaatgtgcacatgctgaaccggataaggacctggcagaaaacccaccagggggagaacaatgggcttgctaacagtatccgggattctttaaatgaatacgaagccaaactcagtgaccttcgtgctcggctgcaggaggcagctgcccaagccaagcaggcaaatggcttgaaccaagaaaacgagagagctttgggagccattcagagacaagtgaaagaaataaattccctgcagagtgatttcaccaagtatctaaccactgcagactcatctttgttgcaaaccaacattgcgctgcagctgatggagaaaagccagaaggaatatgaaaaattagctgccagtttaaatgaagcaagacaagaactaagtgacaaagtaagagaactttccagatctgctggcaaaacatcccttgtggaggaggcagaaaagcacgcgcggtccttacaagagctggcaaagcagctggaagagatcaagagaaacgccagcggggatgagctggtgcgctgtgctgtggatgccgccaccgcctacgagaacatcctcaatgccatcaaagcggccgaggacgcagccaacagggctgccagtgcatctgaatctgccctccagacagtgataaaggaagatctgccaagaaaagctaaaaccctgagttccaacagtgataaactgttaaatgaagccaagatgacacaaaagaagctaaagcaagaagtcagtccagctctcaacaacctacagcaaaccctgaatattgtgacagttcagaaagaagtgatagacaccaatctcacaactctccgagatggtcttcatgggatacagagaggtgatattgatgctatgatcagtagtgcaaagagcatggtcagaaaggccaacgacatcacagatgaggttctggatgggctcaaccccatccagacagatgtggaaagaattaaggacacctatgggaggacacagaacgaagacttcaaaaaggctctgactgatgcagataactcggtgaataagttaaccaacaaactacctgatctttggcgcaagattgaaagtatcaaccaacagctgttgcccttgggaaacatctctgacaacatggacagaatacgagaactaattcagcaggccagagatgctgccagtaaggttgctgtccccatgaggttcaatggtaaatctggagtcgaagtccgactgccaaatgacctggaagatttgaaaggatatacatctctgtccttgtttctccaaaggcccaactcaagagaaaatgggggtactgagaatatgtttgtgatgtaccttggaaataaagatgcctcccgggactacatcggcatggcagttgtggatggccagctcacctgtgtctacaacctgggggaccgtgaggctgaactccaagtggaccagatcttgaccaagagtgagactaaggaggcagttatggatcgggtgaaatttcagagaatttatcagtttgcaaggcttaattacaccaaaggagccacatccagtaaaccagaaacacccggagtctatgacatggatggtagaaatagcaatacactccttaatttggatcctgaaaatgttgtattttatgttggaggttacccacctgattttaaacttcccagtcgactaagtttccctccatacaaaggttgtattgaattagatgacctcaatgaaaatgttctgagcttgtacaacttcaaaaaaacattcaatctcaacacaactgaagtggagccttgtagaaggaggaaggaagagtcagacaaaaattattttgaaggtacgggctatgctcgagttccaactcaaccacatgctcccatcccaacctttggacagacaattcagaccaccgtggatagaggcttgctgttctttgcagaaaacggggatcgcttcatatctctaaatatagaagatggcaagctcatggtgagatacaaactgaattcagagctaccaaaagagagaggagttggagacgccataaacaacggcagagaccattcgattcagatcaaaattggaaaactccaaaagcgtatgtggataaatgtggacgttcaaaacactataattgatggtgaagtatttgatttcagcacatattatctgggaggaattccaattgcaatcagggaaagatttaacatttctacgcctgctttccgaggctgcatgaaaaatttgaagaaaaccagtggtgtcgttagattgaatgatactgtgggagtaaccaaaaagtgctcggaagactggaagcttgtgcgatctgcctcattctccagaggaggacaattgagtttcactgatttgggcttaccacctactgaccacctccaggcctcatttggatttcagacctttcaacccagtggcatattattagatcatcagacatggacaaggaacctgcaggtcactctggaagatggttacattgaattgagcaccagcgatagcggcggcccaatttttaaatctccacagacgtatatggatggtttactgcattatgtatctgtaataagcgacaactctggactacggcttctcatcgatgaccagcttctgagaaatagcaaaaggctaaaacacatttcaagttcccggcagtctctgcgtctgggcgggagcaattttgagggttgtattagcaatgtttttgtccagaggttatcactgagtcctgaagtcctagatttgaccagtaactctctcaagagagatgtgtccctgggaggctgcagtttaaacaaaccaccttttctaatgttgcttaaaggttctaccaggtttaacaagaccaagacttttcgtatcaaccagctgttgcaggacacaccagtggcctccccaaggagcgtgaaggtgtggcaagatgcttgctcaccacttcccaagacccaggccaatcatggagccctccagtttggggacattcccaccagccacttgctattcaagcttcctcaggagctgctgaaacccaggtcacagtttgctgtggacatgcagacaacatcctccagaggactggtgtttcacacgggcactaagaactcctttatggctctttatctttcaaaaggacgtctggtctttgcactggggacagatgggaaaaaattgaggatcaaaagcaaggagaaatgcaatgatgggaaatggcacacggtggtgtttggccatgatggggaaaaggggcgcttggttgtggatggactgagggcccgggagggaagtttgcctggaaactccaccatcagcatcagagcgccagtttacctgggatcacctccatcagggaaaccaaagagcctccccacaaacagctttgtgggatgcctgaagaactttcagctggattcaaaacccttgtataccccttcttcaagcttcggggtgtcttcctgcttgggtggtcctttggagaaaggcatttatttctctgaagaaggaggtcatgtcgtcttggctcactctgtattgttggggccagaatttaagcttgttttcagcatccgcccaagaagtctcactgggatcctaatacacatcggaagtcagcccgggaagcacttatgtgtttacctggaggcaggaaaggtcacggcctctatggacagtggggcaggtgggacctcaacgtcggtcacaccaaagcagtctctgtgtgatggacagtggcactcggtggcagtcaccataaaacaacacatcctgcacctggaactggacacagacagtagctacacagctggacagatccccttcccacctgccagcactcaagagccactacaccttggaggtgctccagccaatttgacgacactgaggatccctgtgtggaaatcattctttggctgtctgaggaatattcatgtcaatcacatccctgtccctgtcactgaagccttggaagtccaggggcctgtcagtctgaatggttgtcctgaccagtaa

SEQ ID NO: 8 - protein, 3333 aa, Homo sapiens
MAAAARPRGRALGPVLPPTPLLLLVLRVLPACGATARDPGAAAGLSLHPTYFNLAEAARIWATATCGERGPGEGRPQPELYCKLVGGPTAPGSGHTIQGQFCDYCNSEDPRKAHPVTNAIDGSERWWQSPPLSSGTQYNRVNLTLDLGQLFHVAYILIKFANSPRPDLWVLERSVDFGSTYSPWQYFAHSKVDCLKEFGREANMAVTRDDDVLCVTEYSRIVPLENGEVVVSLINGRPGAKNFTFSHTLREFTKATNIRLRFLRTNTLLGHLISKAQRDPTVTRRYYYSIKDISIGGQCVCNGHAEVCNINNPEKLFRCECQHHTCGETCDRCCTGYNQRRWRPAAWEQSHECEACNCHGHASNCYYDPDVERQQASLNTQGIYAGGGVCINCQHNTAGVNCEQCAKGYYRPYGVPVDAPDGCIPCSCDPEHADGCEQGSGRCHCKPNFHGDNCEKCAIGYYNFPFCLRIPIFPVSTPSSEDPVAGDIKGCDCNLEGVLPEICDAHGRCLCRPGVEGPRCDTCRSGFYSFPICQACWCSALGSYQMPCSSVTGQCECRPGVTGQRCDRCLSGAYDFPHCQGSSSACDPAGTINSNLGYCQCKLHVEGPTCSRCKLLYWNLDKENPSGCSECKCHKAGTVSGTGECRQGDGDCHCKSHVGGDSCDTCEDGYFALEKSNYFGCQGCQCDIGGALSSMCSGPSGVCQCREHVVGKVCQRPENNYYFPDLHHMKYEIEDGSTPNGRDLRFGFDPLAFPEFSWRGYAQMTSVQNDVRITLNVGKSSGSLFRVILRYVNPGTEAVSGHITIYPSWGAAQSKEIIFLPSKEPAFVTVPGNGFADPFSITPGIWVACIKAEGVLLDYLVLLPRDYYEASVLQLPVTEPCAYAGPPQENCLLYQHLPVTRFPCTLACEARHFLLDGEPRPVAVRQPTPAHPVMVDLSGREVELHLRLRIPQVGHYVVVVEYSTEAAQLFVVDVNVKSSGSVLAGQVNIYSCNYSVLCRSAVIDHMSRIAMYELLADADIQLKGHMARFLLHQVCIIPIEEFSAEYVRPQVHCIASYGRFVNQSATCVSLAHETPPTALILDVLSGRPFPHLPQQSSPSVDVLPGVTLKAPQNQVTLRGRVPHLGRYVFVIHFYQAAHPTFPAQVSVDGGWPRAGSFHASFCPHVLGCRDQVIAEGQIEFDISEPEVAATVKVPEGKSLVLVRVLVVPAENYDYQILHKKSMDKSLEFITNCGKNSFYLDPQTASRFCKNSARSLVAFYHKGALPCECHPTGATGPHCSPEGGQCPCQPNVIGRQCTRCATGHYGFPRCKPCSCGRRLCEEMTGQCRCPPRTVRPQCEVCETHSFSFHPMAGCEGCNCSRRGTIEAAMPECDRDSGQCRCKPRITGRQCDRCASGFYRFPECVPCNCNRDGTEPGVCDPGTGACLCKENVEGTECNVCREGSFHLDPANLKGCTSCFCFGVNNQCHSSHKRRTKFVDMLGWHLETADRVDIPVSFNPGSNSMVADLQELPATIHSASWVAPTSYLGDKVSSYGGYLTYQAKSFGLPGDMVLLEKKPDVQLTGQHMSIIYEETNTPRPDRLHHGRVHVVEGNFRHASSRAPVSREELMTVLSRLADVRIQGLYFTETQRLTLSEVGLEEASDTGSGRIALAVEICACPPAYAGDSCQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHILLKQISGTDGEGNNVPSGDFSREWAEAQRMMRELRNRNFGKHLREAEADKRESQLLLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 9 - DNA, 3336 bp, Homo sapiens
atgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactccggacggtgcagctgtaaaccaggtgtgacaggagccagatgcgaccgatgtctgccaggcttccacatgctcacggatgcggggtgcacccaagaccagagactgctagactccaagtgtgactgtgacccagctggcatcgcagggccctgtgacgcgggccgctgtgtctgcaagccagctgtcactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagaccctgtctattttgtggctcctgccaaatttcttgggaatcaacaggtgagctatggtcaaagcctgtcctttgactaccgtgtggacagaggaggcagacacccatctgcccatgatgtgattctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgatgccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacaacaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctcggcttcagggagtcagtgatcagtcctttcaggtggaagaagcaaagaggatcaaacaaaaagcggattcactctcaagcctggtaaccaggcatatggatgagttcaagcgtacacagaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatgggtatgtga

SEQ ID NO: 10 - protein, 1111 aa, Homo sapiens
MPALWLGCCLCFSLLLPAARATSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMGM

(19) United States
(12) Patent Application Publication
(10) Pub. No.: US 2020/0291353 A1
(43) Pub. Date: Sep. 17, 2020

(54) METHOD FOR PRODUCING CELL FLAPS

(71) Applicants: HOLOSTEM TERAPIE AVANZATE S.R.L., Modena (MO) (IT); UNIVERSITA' DEGLI STUDI DI MODENA E REGGIO EMILIA, Modena (MO) (IT)

(72) Inventors: Michele DE LUCA, Modena (MO) (IT); Graziella PELLEGRINI, Modena (MO) (IT); Andrea ALESSANDRINI, Modena (MO) (IT)

(73) Assignee: HOLOSTEM TERAPIE AVANZATE S.R.L., Modena (MO) (IT)

(21) Appl. No.: 16/639,955

(22) PCT Filed: Aug. 10, 2018
(86) PCT No.: PCT/EP2018/071736

§ 371 Date: Feb. 18, 2020

(30) Foreign Application Priority Data
Aug. 17, 2017 (IT) 102017000094210

(51) Int. Cl. C12N 5/071; C12N 15/86

(52) U.S. Cl. CPC C12N 5/0629; C12N 15/86; C12N 2740/10043; C12N 2510/00; C12N 2533/56

(57) ABSTRACT

The present invention refers to an in vitro method for producing a flap of genetically modified cells on fibrin substrate and to the flap so obtained.

### FIELD OF THE INVENTION

The present invention refers to an in vitro or ex vivo method for producing a flap of genetically modified cells on fibrin substrate and the flap so obtained.

### BACKGROUND ART

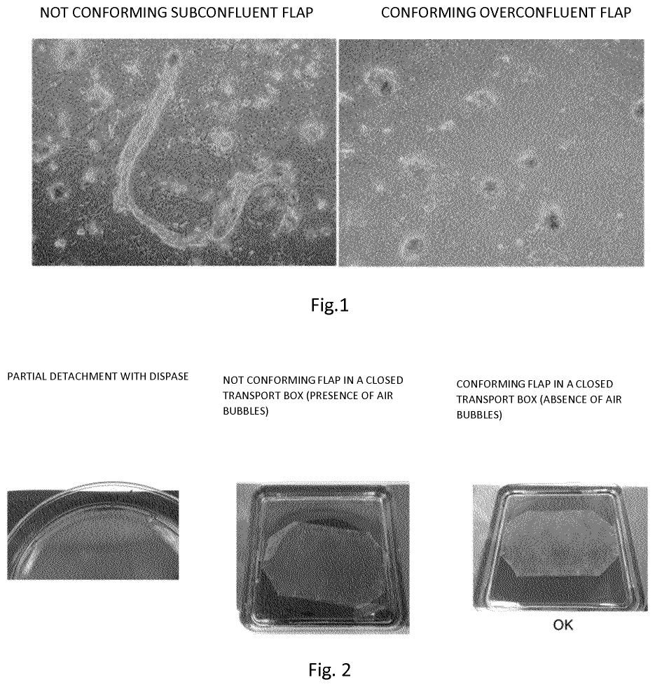

To date, the procedure for the preparation of ex vivo genetically modified epidermis flaps involves the culture of cells on plastic supports with the aim of obtaining a genetically modified flap of the epidermis. The procedure described to date, for example in Mavilio et al. 2006⁽¹²⁾ and Bauer et al. 2017⁽¹⁰⁾, consists in plating, on plastic supports of 75-175 cm², keratinocytes genetically corrected on feeder layers and allowing them to grow and reach full confluence (9-14 days). The attainment of the confluence represents a fundamental step to ensure the stability of the flap (FIG. 1). The reason is due to the intrinsic stratification/differentiation process in keratinocytes, which, once they reach the confluence, slow down their proliferation in favor of stratification/differentiation processes. The stratification process ensures greater stability and compactness on the flap of the epidermis so formed, thus ensuring better maneuverability, a condition necessary for the assembly and transportation phases. Upon reaching the confluence (FIG. 1), the epidermis flap is washed with a solution containing DMEM, L-Glutamine. Subsequently, the flap is dissociated from the plastic support by the addition of Dispase II (2.5 mg/ml). On the upper side (opposite to the one adhering to plastic) a Vaseline® Petrolatum gauze of 50 cm² is applied, which will be fixed to the epidermis flap by clips. Once the flap is secured, this is transferred to a transport flap container (or transportation box) (FIG. 2).

The method for obtaining a flap starting from a plastic support is a long and complicated procedure. There are several steps that may invalidate the release of the same. The following table show the main steps that may lead to the non-conformity of the flap and to the loss of release (Table 1).

TABLE 1
Parameters of non-conformity in releasing the flap.

| | Not |
|---|---|
| Parameters | conform |
| No conform evaluation of culture confluence before detachment | x |
| Presence of breaks after DISPASE detachment | x |
| Presence of breaks after application of gauzes and clips | x |
| Presence of bubbles in the transportation box | x |
| Presence of breaks during shipment | x |
| Presence of breaks after the transportation box opening | x |

Regardless of the procedure used for preparing the flap, another step that can be a cause of failure to release the flap is its breakup during the transportation phases. Although the flap is secured and locked on the gauze, unintentional movements during transport may cause its breakage or the winding on itself. In case of breakage, the flap is considered to be inadequate (Table 1).

The procedure described above is not intuitive and without risk. In fact, in the setup phases, given the multiple steps and continuous manipulations, the risk of contamination or the presence of air bubbles, which can alter the O₂ exchange, may result in poor product quality. It is also known that keratinocytes in the absence of adhesion induce the activation of terminal differentiation processes (Watt F M, Jordan P W, O'Neill C H. 1988. Cell shape controls terminal differentiation of human epidermal keratinocytes. Proc Natl Acad Sci USA 85:5576-5580), in fact, the stability of the genetically modified flap generated from plastic supports is 24 h. The biological quality as well as the performance of the flap so produced are remarkably reduced after 24 hours. Therefore, is still felt the need of an alternative method for providing genetically modified flaps wherein the cells are not subjected to an accelerated terminal differentiation due to the loss of contact with the substrate which usually happens in the previous disclosed conditions.

The patent application WO2005028638 refers to a process for producing a cell sheet, comprising culturing cells up to a state of saturation on the surface of a support having its surface coated with fibrin, continuing the culturing for a period of time sufficient to achieve decomposition of the fibrin at cell bottom surface and detaching the cultured cells in the form of a sheet from the support surface. Therefore, the patent application WO2005028638 teaches to obtain a sheet of cells not genetically modified, wherein the fibrin is not present because it was previously degraded.

A paper published by Pellegrini et al. in 1999, show the potential use of a matrix of fibrin for culturing human epithelial stem cells. Said publication shows that the culture of human keratinocytes on fibrin doesn't alter the biological properties of the cells and maintains their characteristic of staminality, as demonstrated by the presence of isolated holoclones in these conditions (epidermal stem cells) (Gallico, G. G., 3rd, et al. Permanent coverage of large burn wounds with autologous cultured human epithelium. The New England journal of medicine 311, 448-451 (1984); Pellegrini, G. et al. The control of epidermal stem cells (holoclones) in the treatment of massive full-thickness burns with autologous keratinocytes cultured on fibrin. Transplantation 68, 868-879 (1999); Cuono C. et al. Use of cultured epidermal autografts and dermal allografts as skin replacement after burn injury. Lancet 1:1123-1124 (1986); De Luca M, et al. 1989. Multicentre experience in the treatment of burns with autologous and allogenic cultured epithelium, fresh or preserved in a frozen state. Burns 15:303-309). In 2010, another work was published that demonstrates the clinical effectiveness of transplant of Corneal limbal cells in the treatment of severe burns by corneal epithelium (Rama P, Matuska S, Paganoni G, Spinelli A, De Luca M, Pellegrini G. 2010. Limbal stem-cell therapy and long-term corneal regeneration. N Engl J Med 363:147-155). In both papers, the epithelium was cultivated on a fibrin matrix starting from raw materials (fibrinogen and thrombin) produced, for example, by Baxter (Tissucol). This fibrin has been used in more than 200 epithelial corneal cell transplants, none of which has been found to have any adverse events due to rejection or inflammation. Preferably, the fibrin matrix is produced by Holostem Terapie Avanzate, from raw materials (fibrinogen and thrombin) produced for example by Kedrion. A comparative study performed on corneal limbal epithelial cells showed the equivalence of the two products (Table 2).

TABLE 2

| | |
|---|---|
| lots of TISSUCOL | lots of KEDRION |

| parameter | average | dev stand. | average | dev stand. |
|---|---|---|---|---|
| % CFE | 19.1 | 8.1 | 17.6 | 7.9 |
| % Ab | 14.3 | 4.3 | 20.6 | 12.3 |
| % K3 | 86.9 | 2.4 | 86.7 | 5.4 |
| % K19 | 15.5 | 16.3 | 52.7 | 25.8 |
| % p63 | 1.3 | 0.9 | 2.2 | 1 |

Table 2. Resumes the results obtained from a comparative study carried out starting from different fibrin lots, using excipients (fibrin and fibrinogen) produced by Baxter (Tissucol) and by Kedrion. The results obtained show the equivalence of both products, as evidenced by chlonogenic values (% CFE) almost unchanged and by the value of the percentage of p63 positive cells superior when using excipients from Kedrion.

The potential use of a fibrin substrate for culturing cells is therefore already described. However, its use as a substrate for culturing genetically modified cells was not previously disclosed.

It is still felt the need of a method which allows to obtain a flap of genetically modified cells which overcomes the disadvantages of methods which require cell culture on plastic supports.

### SUMMARY OF THE INVENTION

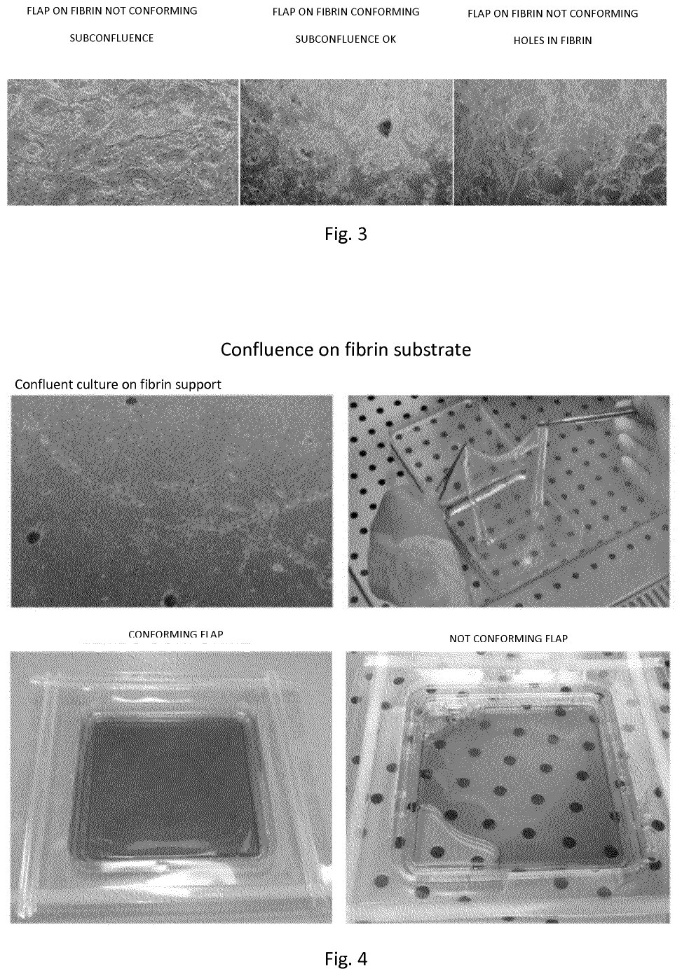

The present inventors have developed a method which uses fibrin for the production of genetically modified cell flaps. In a preferred aspect of the invention, the method comprises plating genetically corrected keratinocytes and feeder layer on a matrix (or substrate) of fibrin of size of 144 cm². Unlike the production process of the epidermis flaps on plastic supports, the keratinocytes cultivated under these conditions do not have to reach full confluence, but the subconfluence to proceed with the preparation of this for transport (FIG. 3).

Fibrin provides growth support to keratinocytes, both in the transport phase and before detaching from the support, thus securing a high proliferative/regenerative potential of the keratinocytes.

This prevents an accelerated differentiation process due to contact loss, found in the epidermis flap derived from growth in plastic (Table 1). During the transport phases, and after the detachment of the flap, (including cells, for example of epidermis, and fibrin), cells, particularly keratinocytes, will complete their growth and begin the in vivo layering/differentiation process. Despite the greater flexibility and handling of fibrin in the transport phase, it is still necessary to perform the compliance checks before the release of the flap. As shown in FIG. 3, holes in the fibrin or disomogeneity in the keratinocyte or feeder plating make the flap non-conforming to release. Fibrin is an ideal support for the growth of keratinocytes because it represents a compact and solid biodegradable biological matrix that ensures a great deal of maneuverability during preparation and transport phases.

The fibrin flap obtained is washed with a solution containing DMEM and L-Glutamine. Then, by means of sterile pliers, it is detached from the holder and placed in the transport container (FIG. 4), where the transport medium will be added. The container is then sealed ensuring that no air bubble is present. The presence of bubbles would render the flap release not adequate for therapeutic applications, such as transplants (FIG. 4).

Unlike the flap derived from growth on plastic, the fracture of the flap on the fibrin during the transport phases is a very rare event.

In addition, the performance and biological stability of the product are superior if compared to flap resulting from growth on plastic support.

In addition to this, the reduced risk of microbiological contamination due to the small number of manipulations required to set up the flap and the reduced risk of breakages in transport should also be taken into account.

The present invention thus allows to obtain cellular flaps from different cell types using the same procedure without any particular modification. In addition, this method allows to obtain a large number of flaps quickly and without the need to use expensive culturing plates.

### DETAILED DESCRIPTION OF THE INVENTION

It is therefore an embodiment of the invention an in vitro method for producing a flap of genetically modified cells on fibrin substrate, characterized by:
a) plating feeder cells on the upper surface of a fibrin substrate so as to obtain a fibrin substrate on which said feeder cells are adhered;
b) plating and cultivating to subconfluence said genetically modified cells on said fibrin substrate onto which feeder cells are adhered, said fibrin substrate being positioned on a solid support so that the cells do not interact with the surface of said support so as to obtain a flap of genetically modified cells adhered to said fibrin substrate;
c) detaching the flap of genetically modified cells adhered to said fibrin substrate from the support in a form similar to a sheet to obtain a flap of genetically modified cells on fibrin substrate.

Said solid support is preferably of plastic, e.g. a Petri dish, or of glass.

Preferably, said feeder cells are plated on the fibrin substrate from 2 to 24 hours before plating the genetically modified cells.

In a preferred embodiment of the invention, the method further comprises: before step c), the steps:
b′) removing the culture medium and/or
b″) washing the flap of genetically modified cells adhered to said fibrin substrate with a washing solution
and/or after step c), the step of:
d) placing the obtained flap of genetically modified cells on fibrin substrate in a transport container.

The transport container preferably comprises a transport medium.

Said fibrin substrate has preferably dimensions of from about 0.32 cm² to about 300 cm², preferably of about 31-144 cm², more preferably of 144 cm².

Preferably, said fibrin substrate comprises from about 20 to about 100 mg/ml of fibrinogen and from about 1 to about 10 IU/ml of thrombin. More preferably, said fibrin substrate comprises from about 20 to about 50 mg/ml of fibrinogen, preferably from about 20 to about 40 mg/ml of fibrinogen, and from about 3 to about 8 IU/ml of thrombin; even more preferably it comprises from about 20 to about 25 mg/ml of fibrinogen and from about 2 to about 4 IU/ml of thrombin. In a preferred aspect said fibrin substrate comprises about 23.1 mg/ml of fibrinogen and about 3.1 IU/ml of thrombin.

Preferably, said genetically modified cells are epithelial cells, preferably primary epithelial cells deriving from stratified epithelia, more preferably epidermal cells, preferably keratinocytes, more preferably human primary keratinocytes isolated from biopsies, preferably skin biopsies. Said genetically modified cells have preferably been transduced with a gene or a cDNA selected from the group consisting of:

a) at least one chain selected from the group consisting of: beta-3, α3 and γ2 chain of laminin-5, and/or

b) collagen 17 and/or

c) at least one α6β4 integrin and/or

d) collagen 7 and/or

e) keratin 5 and Keratin 14 and/or

f) Plectin.

Preferably, said genetically modified cells have preferably been transduced with a gene or a cDNA selected from the group consisting of: beta-3 chain of laminin 5, collagen 7 and collagen 17.

In a preferred embodiment, the gene or cDNA encode for the above-mentioned protein or for an amino acid sequence having at least 75% amino acid sequence identity to the amino acid sequence SEQ ID NO: 6 and/or to the amino acid sequence SEQ ID NO:4 and/or to the amino acid sequence SEQ ID NO: 2.

Another embodiment of the invention is a flap of genetically modified cells on fibrin substrate, obtainable by the above described method.

A further object of the invention is a flap of genetically modified cells on fibrin substrate, wherein said cells are preferably epithelial cells, preferably primary epithelial cells deriving from stratified epithelia, more preferably epidermal cells, preferably keratinocytes, even more preferably human primary keratinocytes isolated from biopsies, preferably skin biopsies.

Preferably said genetically modified cells are transduced with a gene or cDNA selected from the group consisting of:

a) at least one chain selected from the group consisting of: beta-3, α3 and γ2 chain of laminin-5, and/or

b) collagen 17 and/or

c) at least one a6134 integrin and/or

d) collagen 7 and/or

e) keratin 5 and Keratin 14 and/or

f) Plectin.

Preferably said genetically modified cells are transduced with a gene or cDNA selected from the group consisting of: beta-3 chain of laminin 5, collagen 7 and collagen 17.

In a preferred embodiment, the gene or cDNA encode for the above-mentioned protein or for an amino acid sequence having at least 75% amino acid sequence identity to the amino acid sequence SEQ ID NO: 6 and/or to the amino acid sequence SEQ ID NO:4 and/or to the amino acid sequence SEQ ID NO: 2.

Preferably, the genetically modified cells are cells that have been transduced with a retroviral vector, said retroviral vector preferably being an alpharetroviral vector, a gammaretroviral vector, a lentiviral vector or a spumaretroviral vector.

Preferably, said fibrin substrate comprises from about 20 to about 100 mg/ml of fibrinogen and from about 1 to about 10 IU/ml of thrombin. More preferably, said fibrin substrate comprises from about 20 to about 50 mg/ml of fibrinogen, preferably from about 20 to about 40 mg/ml of fibrinogen, and from about 3 to about 8 IU/ml of thrombin; even more preferably it comprises from about 20 to about 25 mg/ml of fibrinogen and from about 2 to about 4 IU/ml of thrombin. In a preferred aspect said fibrin substrate comprises about 23.1 mg/ml of fibrinogen and about 3.1 IU/ml of thrombin.

Another embodiment of the invention is the above described flap for medical use.

A further embodiment of the invention is the use of a solid support which surface is covered by a fibrin substrate for the preparation of flaps of genetically modified cells on fibrin substrate, preferably said fibrin substrate comprises from about 20 to about 100 mg/ml of fibrinogen and from about 1 to about 10 IU/ml of thrombin. More preferably, said fibrin substrate comprises from about 20 to about 50 mg/ml of fibrinogen, preferably from about 20 to about 40 mg/ml of fibrinogen, and from about 3 to about 8 IU/ml of thrombin; even more preferably it comprises from about 20 to about 25 mg/ml of fibrinogen and from about 2 to about 4 IU/ml of thrombin. In a preferred aspect said fibrin substrate comprises about 23.1 mg/ml of fibrinogen and about 3.1 IU/ml of thrombin.

In a preferred embodiment of the invention above disclosed, thawed genetically modified cells, in particular keratinocytes cells, and feeder cells may be plated at the same time. Alternatively, it is possible to plate feeder cells and after 2 h-24 h thawing the genetically modified cells, in particular keratinocytes.

In the context of the present invention “IU” refers to “International Unit”.

In a preferred embodiment of the present invention, the genetically modified cells are cells that have been transduced with a retroviral vector carrying the cDNA of (or the nucleotide sequence encoding for) the beta-3 chain of laminin 5. However, results similar to those herein shown were obtained with similar products (e.g. retroviral vectors carrying different genes). The retroviral vector may e.g. be an alpharetroviral vector, a gammaretroviral vector, a lentiviral vector or a spumaretroviral vector.

In the context of the present invention the “feeder cells” or “feeder” are preferably cells obtained according to the method disclosed in Rheinwald J G, Green H. 1975. Serial cultivation of strains of human epidermal keratinocytes: the formation of keratinizing colonies from single cells. Cell 6:331-343.

They correspond to a clone of murine cells isolated in the laboratory of prof. Green H. (Rheinwald, J. et al 1975).

With the term “flap of cells” or “cell flap” it is intended preferably a sheet of epithelial cells, comprising cells in a single layer or in multilayer able to recreate an epidermis ex vivo.

According to the present invention the fibrin substrate (or fibrin support) is preferably a fibrin gel which is obtainable by admixing fibrinogen and thrombin, thus obtaining a fibrinogen and thrombin composition or solution.

The step of detachment of the flap from the support in the method according to the present invention is preferably carried out by mechanical methods, e.g. using pliers or forceps. However, any method known by the skilled man may be used.

In the context of the present invention “similar to a sheet” is preferably intended as an intact cell sheet.

The term “comprises” when referred to the fibrin substrate can also be intended as “obtainable by admixing”.

The expression “genetically modified cells” includes cells comprising a heterologous nucleic acid, for example which were transduced or transfected with one or more nucleic acid.

Said heterologous nucleic acid is preferably at least one gene or cDNA (or a nucleotide sequence encoding for a polypeptide) selected from the group consisting of: beta-3 chain of laminin 5, collagen 7, collagen 17 or combination thereof.

The starting cell may be e.g. transduced or transfected with a construct that will be integrated in the cell genome in place of the target endogenous gene or in different regions, where said construct comprises a heterologous sequence of the gene of interest and in some cases also a selectable marker which allows to select the obtained genetically modified cells. Alternatively, the genetically modified cells may not comprise a sequence (also partial) of a particular nucleic acid encoding a specific protein or peptide, for example obtained by deletion of a genetic sequence. The washing solution used in the above method is preferably “Dulbecco's modified eagle medium (DMEM)”, supplemented with L-glutamine. The transport medium used in the above method is preferably “Dulbecco's modified eagle medium (DMEM)”, supplemented with L-glutamine.

Preferably, the collagen 7 is characterized by the sequence as disclosed in in the NCBI Data Bank with the Accession no.: NM_000094.3 (Col7A1). The cDNA sequence is:

(SEQ ID NO: 1)
ATGACGCTGCGGCTTCTGGTGGCCGCGCTCTGCGCCGGGATCCTGGCAGA
GGCGCCCCGAGTGCGAGCCCAGCACAGGGAGAGAGTGACCTGCACGCGCC
TTTACGCCGCTGACATTGTGTTCTTACTGGATGGCTCCTCATCCATTGGC
CGCAGCAATTTCCGCGAGGTCCGCAGCTTTCTCGAAGGGCTGGTGCTGCC
TTTCTCTGGAGCAGCCAGTGCACAGGGTGTGCGCTTTGCCACAGTGCAGT
ACAGCGATGACCCACGGACAGAGTTCGGCCTGGATGCACTTGGCTCTGGG
GGTGATGTGATCCGCGCCATCCGTGAGCTTAGCTACAAGGGGGGCAACAC
TCGCACAGGGGCTGCAATTCTCCATGTGGCTGACCATGTCTTCCTGCCCC
AGCTGGCCCGACCTGGTGTCCCCAAGGTCTGCATCCTGATCACAGACGGG
AAGTCCCAGGACCTGGTGGACACAGCTGCCCAAAGGCTGAAGGGGCAGGG
GGTCAAGCTATTTGCTGTGGGGATCAAGAATGCTGACCCTGAGGAGCTGA
AGCGAGTTGCCTCACAGCCCACCAGTGACTTCTTCTTCTTCGTCAATGAC
TTCAGCATCTTGAGGACACTACTGCCCCTCGTTTCCCGGAGAGTGTGCAC
GACTGCTGGTGGCGTGCCTGTGACCCGACCTCCGGATGACTCGACCTCTG
CTCCACGAGACCTGGTGCTGTCTGAGCCAAGCAGCCAATCCTTGAGAGTA
CAGTGGACAGCGGCCAGTGGCCCTGTGACTGGCTACAAGGTCCAGTACAC
TCCTCTGACGGGGCTGGGACAGCCACTGCCGAGTGAGCGGCAGGAGGTGA
ACGTCCCAGCTGGTGAGACCAGTGTGCGGCTGCGGGGTCTCCGGCCACTG
ACCGAGTACCAAGTGACTGTGATTGCCCTCTACGCCAACAGCATCGGGGA
GGCTGTGAGCGGGACAGCTCGGACCACTGCCCTAGAAGGGCCGGAACTGA
CCATCCAGAATACCACAGCCCACAGCCTCCTGGTGGCCTGGCGGAGTGTG
CCAGGTGCCACTGGCTACCGTGTGACATGGCGGGTCCTCAGTGGTGGGCC
CACACAGCAGCAGGAGCTGGGCCCTGGGCAGGGTTCAGTGTTGCTGCGTG
ACTTGGAGCCTGGCACGGACTATGAGGTGACCGTGAGCACCCTATTTGGC
CGCAGTGTGGGGCCCGCCACTTCCCTGATGGCTCGCACTGACGCTTCTGT
TGAGCAGACCCTGCGCCCGGTCATCCTGGGCCCCACATCCATCCTCCTTT
CCTGGAACTTGGTGCCTGAGGCCCGTGGCTACCGGTTGGAATGGCGGCGT
GAGACTGGCTTGGAGCCACCGCAGAAGGTGGTACTGCCCTCTGATGTGAC
CCGCTACCAGTTGGATGGGCTGCAGCCGGGCACTGAGTACCGCCTCACAC
TCTACACTCTGCTGGAGGGCCACGAGGTGGCCACCCCTGCAACCGTGGTT
CCCACTGGACCAGAGCTGCCTGTGAGCCCTGTAACAGACCTGCAAGCCAC
CGAGCTGCCCGGGCAGCGGGTGCGAGTGTCCTGGAGCCCAGTCCCTGGTG
CCACCCAGTACCGCATCATTGTGCGCAGCACCCAGGGGGTTGAGCGGACC
CTGGTGCTTCCTGGGAGTCAGACAGCATTCGACTTGGATGACGTTCAGGC
TGGGCTTAGCTACACTGTGCGGGTGTCTGCTCGAGTGGGTCCCCGTGAGG
GCAGTGCCAGTGTCCTCACTGTCCGCCGGGAGCCGGAAACTCCACTTGCT
GTTCCAGGGCTGCGGGTTGTGGTGTCAGATGCAACGCGAGTGAGGGTGGC
CTGGGGACCCGTCCCTGGAGCCAGTGGATTTCGGATTAGCTGGAGCACAG
GCAGTGGTCCGGAGTCCAGCCAGACACTGCCCCCAGACTCTACTGCCACA
GACATCACAGGGCTGCAGCCTGGAACCACCTACCAGGTGGCTGTGTCGGT
ACTGCGAGGCAGAGAGGAGGGCCCTGCTGCAGTCATCGTGGCTCGAACGG
ACCCACTGGGCCCAGTGAGGACGGTCCATGTGACTCAGGCCAGCAGCTCA
TCTGTCACCATTACCTGGACCAGGGTTCCTGGCGCCACAGGATACAGGGT
TTCCTGGCACTCAGCCCACGGCCCAGAGAAATCCCAGTTGGTTTCTGGGG
AGGCCACGGTGGCTGAGCTGGATGGACTGGAGCCAGATACTGAGTATACG
GTGCATGTGAGGGCCCATGTGGCTGGCGTGGATGGGCCCCCTGCCTCTGT
GGTTGTGAGGACTGCCCCTGAGCCTGTGGGTCGTGTGTCGAGGCTGCAGA
TCCTCAATGCTTCCAGCGACGTTCTACGGATCACCTGGGTAGGGGTCACT
GGAGCCACAGCTTACAGACTGGCCTGGGGCCGGAGTGAAGGCGGCCCCAT
GAGGCACCAGATACTCCCAGGAAACACAGACTCTGCAGAGATCCGGGGTC
TCGAAGGTGGAGTCAGCTACTCAGTGCGAGTGACTGCACTTGTCGGGGAC
CGCGAGGGCACACCTGTCTCCATTGTTGTCACTACGCCGCCTGAGGCTCC
GCCAGCCCTGGGGACGCTTCACGTGGTGCAGCGCGGGGAGCACTCGCTGA
GGCTGCGCTGGGAGCCGGTGCCCAGAGCGCAGGGCTTCCTTCTGCACTGG
CAACCTGAGGGTGGCCAGGAACAGTCCCGGGTCCTGGGGCCCGAGCTCAG
CAGCTATCACCTGGACGGGCTGGAGCCAGCGACACAGTACCGCGTGAGGC
TGAGTGTCCTAGGGCCAGCTGGAGAAGGGCCCTCTGCAGAGGTGACTGCG
CGCACTGAGTCACCTCGTGTTCCAAGCATTGAACTACGTGTGGTGGACAC
CTCGATCGACTCGGTGACTTTGGCCTGGACTCCAGTGTCCAGGGCATCCA
GCTACATCCTATCCTGGCGGCCACTCAGAGGCCCTGGCCAGGAAGTGCCT
GGGTCCCCGCAGACACTTCCAGGGATCTCAAGCTCCCAGCGGGTGACAGG
GCTAGAGCCTGGCGTCTCTTACATCTTCTCCCTGACGCCTGTCCTGGATG
GTGTGCGGGGTCCTGAGGCATCTGTCACACAGACGCCAGTGTGCCCCCGT
GGCCTGGCGGATGTGGTGTTCCTACCACATGCCACTCAAGACAATGCTCA
CCGTGCGGAGGCTACGAGGAGGGTCCTGGAGCGTCTGGTGTTGGCACTTG
GGCCTCTTGGGCCACAGGCAGTTCAGGTTGGCCTGCTGTCTTACAGTCAT
CGGCCCTCCCCACTGTTCCCACTGAATGGCTCCCATGACCTTGGCATTAT
CTTGCAAAGGATCCGTGACATGCCCTACATGGACCCAAGTGGGAACAACC
TGGGCACAGCCGTGGTCACAGCTCACAGATACATGTTGGCACCAGATGCT
CCTGGGCGCCGCCAGCACGTACCAGGGGTGATGGTTCTGCTAGTGGATGA
ACCCTTGAGAGGTGACATATTCAGCCCCATCCGTGAGGCCCAGGCTTCTG
GGCTTAATGTGGTGATGTTGGGAATGGCTGGAGCGGACCCAGAGCAGCTG
CGTCGCTTGGCGCCGGGTATGGACTCTGTCCAGACCTTCTTCGCCGTGGA
TGATGGGCCAAGCCTGGACCAGGCAGTCAGTGGTCTGGCCACAGCCCTGT
GTCAGGCATCCTTCACTACTCAGCCCCGGCCAGAGCCCTGCCCAGTGTAT
TGTCCAAAGGGCCAGAAGGGGGAACCTGGAGAGATGGGCCTGAGAGGACA
AGTTGGGCCTCCTGGCGACCCTGGCCTCCCGGGCAGGACCGGTGCTCCCG
GCCCCCAGGGGCCCCCTGGAAGTGCCACTGCCAAGGGCGAGAGGGGCTTC
CCTGGAGCAGATGGGCGTCCAGGCAGCCCTGGCCGCGCCGGGAATCCTGG
GACCCCTGGAGCCCCTGGCCTAAAGGGCTCTCCAGGGTTGCCTGGCCCTC
GTGGGGACCCGGGAGAGCGAGGACCTCGAGGCCCAAAGGGGGAGCCGGGG
GCTCCCGGACAAGTCATCGGAGGTGAAGGACCTGGGCTTCCTGGGCGGAA
AGGGGACCCTGGACCATCGGGCCCCCCTGGACCTCGTGGACCACTGGGGG
ACCCAGGACCCCGTGGCCCCCCAGGGCTTCCTGGAACAGCCATGAAGGGT
GACAAAGGCGATCGTGGGGAGCGGGGTCCCCCTGGACCAGGTGAAGGTGG
CATTGCTCCTGGGGAGCCTGGGCTGCCGGGTCTTCCCGGAAGCCCTGGAC
CCCAAGGCCCCGTTGGCCCCCCTGGAAAGAAAGGAGAAAAAGGTGACTCT
GAGGATGGAGCTCCAGGCCTCCCAGGACAACCTGGGTCTCCGGGTGAGCA
GGGCCCACGGGGACCTCCTGGAGCTATTGGCCCCAAAGGTGACCGGGGCT
TTCCAGGGCCCCTGGGTGAGGCTGGAGAGAAGGGCGAACGTGGACCCCCA
GGCCCAGCGGGATCCCGGGGGCTGCCAGGGGTTGCTGGACGTCCTGGAGC
CAAGGGTCCTGAAGGGCCACCAGGACCCACTGGCCGCCAAGGAGAGAAGG
GGGAGCCTGGTCGCCCTGGGGACCCTGCAGTGGTGGGACCTGCTGTTGCT
GGACCCAAAGGAGAAAAGGGAGATGTGGGGCCCGCTGGGCCCAGAGGAGC
TACCGGAGTCCAAGGGGAACGGGGCCCACCCGGCTTGGTTCTTCCTGGAG
ACCCTGGCCCCAAGGGAGACCCTGGAGACCGGGGTCCCATTGGCCTTACT
GGCAGAGCAGGACCCCCAGGTGACTCAGGGCCTCCTGGAGAGAAGGGAGA
CCCTGGGCGGCCTGGCCCCCCAGGACCTGTTGGCCCCCGAGGACGAGATG
GTGAAGTTGGAGAGAAAGGTGACGAGGGTCCTCCGGGTGACCCGGGTTTG
CCTGGAAAAGCAGGCGAGCGTGGCCTTCGGGGGGCACCTGGAGTTCGGGG
GCCTGTGGGTGAAAAGGGAGACCAGGGAGATCCTGGAGAGGATGGACGAA
ATGGCAGCCCTGGATCATCTGGACCCAAGGGTGACCGTGGGGAGCCGGGT
CCCCCAGGACCCCCGGGACGGCTGGTAGACACAGGACCTGGAGCCAGAGA
GAAGGGAGAGCCTGGGGACCGCGGACAAGAGGGTCCTCGAGGGCCCAAGG
GTGATCCTGGCCTCCCTGGAGCCCCTGGGGAAAGGGGCATTGAAGGGTTT
CGGGGACCCCCAGGCCCACAGGGGGACCCAGGTGTCCGAGGCCCAGCAGG
AGAAAAGGGTGACCGGGGTCCCCCTGGGCTGGATGGCCGGAGCGGACTGG
ATGGGAAACCAGGAGCCGCTGGGCCCTCTGGGCCGAATGGTGCTGCAGGC
AAAGCTGGGGACCCAGGGAGAGACGGGCTTCCAGGCCTCCGTGGAGAACA
GGGCCTCCCTGGCCCCTCTGGTCCCCCTGGATTACCGGGAAAGCCAGGCG
AGGATGGCAAACCTGGCCTGAATGGAAAAAACGGAGAACCTGGGGACCCT
GGAGAAGACGGGAGGAAGGGAGAGAAAGGAGATTCAGGCGCCTCTGGGAG
AGAAGGTCGTGATGGCCCCAAGGGTGAGCGTGGAGCTCCTGGTATCCTTG
GACCCCAGGGGCCTCCAGGCCTCCCAGGGCCAGTGGGCCCTCCTGGCCAG
GGTTTTCCTGGTGTCCCAGGAGGCACGGGCCCCAAGGGTGACCGTGGGGA
GACTGGATCCAAAGGGGAGCAGGGCCTCCCTGGAGAGCGTGGCCTGCGAG
GAGAGCCTGGAAGTGTGCCGAATGTGGATCGGTTGCTGGAAACTGCTGGC
ATCAAGGCATCTGCCCTGCGGGAGATCGTGGAGACCTGGGATGAGAGCTC
TGGTAGCTTCCTGCCTGTGCCCGAACGGCGTCGAGGCCCCAAGGGGGACT
CAGGCGAACAGGGCCCCCCAGGCAAGGAGGGCCCCATCGGCTTTCCTGGA
GAACGCGGGCTGAAGGGCGACCGTGGAGACCCTGGCCCTCAGGGGCCACC
TGGTCTGGCCCTTGGGGAGAGGGGCCCCCCCGGGCCTTCCGGCCTTGCCG
GGGAGCCTGGAAAGCCTGGTATTCCCGGGCTCCCAGGCAGGGCTGGGGGT
GTGGGAGAGGCAGGAAGGCCAGGAGAGAGGGGAGAACGGGGAGAGAAAGG
AGAACGTGGAGAACAGGGCAGAGATGGCCCTCCTGGACTCCCTGGAACCC
CTGGGCCCCCCGGACCCCCTGGCCCCAAGGTGTCTGTGGATGAGCCAGGT
CCTGGACTCTCTGGAGAACAGGGACCCCCTGGACTCAAGGGTGCTAAGGG
GGAGCCGGGCAGCAATGGTGACCAAGGTCCCAAAGGAGACAGGGGTGTGC
CAGGCATCAAAGGAGACCGGGGAGAGCCTGGACCGAGGGGTCAGGACGGC
AACCCGGGTCTACCAGGAGAGCGTGGTATGGCTGGGCCTGAAGGGAAGCC
GGGTCTGCAGGGTCCAAGAGGCCCCCCTGGCCCAGTGGGTGGTCATGGAG
ACCCTGGACCACCTGGTGCCCCGGGTCTTGCTGGCCCTGCAGGACCCCAA
GGACCTTCTGGCCTGAAGGGGGAGCCTGGAGAGACAGGACCTCCAGGACG
GGGCCTGACTGGACCTACTGGAGCTGTGGGACTTCCTGGACCCCCCGGCC
CTTCAGGCCTTGTGGGTCCACAGGGGTCTCCAGGTTTGCCTGGACAAGTG
GGGGAGACAGGGAAGCCGGGAGCCCCAGGTCGAGATGGTGCCAGTGGAAA
AGATGGAGACAGAGGGAGCCCTGGTGTGCCAGGGTCACCAGGTCTGCCTG
GCCCTGTCGGACCTAAAGGAGAACCTGGCCCCACGGGGGCCCCTGGACAG
GCTGTGGTCGGGCTCCCTGGAGCAAAGGGAGAGAAGGGAGCCCCTGGAGG
CCTTGCTGGAGACCTGGTGGGTGAGCCGGGAGCCAAAGGTGACCGAGGAC
TGCCAGGGCCGCGAGGCGAGAAGGGTGAAGCTGGCCGTGCAGGGGAGCCC
GGAGACCCTGGGGAAGATGGTCAGAAAGGGGCTCCAGGACCCAAAGGTTT
CAAGGGTGACCCAGGAGTCGGGGTCCCGGGCTCCCCTGGGCCTCCTGGCC
CTCCAGGTGTGAAGGGAGATCTGGGCCTCCCTGGCCTGCCCGGTGCTCCT
GGTGTTGTTGGGTTCCCGGGTCAGACAGGCCCTCGAGGAGAGATGGGTCA
GCCAGGCCCTAGTGGAGAGCGGGGTCTGGCAGGCCCCCCAGGGAGAGAAG
GAATCCCAGGACCCCTGGGGCCACCTGGACCACCGGGGTCAGTGGGACCA
CCTGGGGCCTCTGGACTCAAAGGAGACAAGGGAGACCCTGGAGTAGGGCT
GCCTGGGCCCCGAGGCGAGCGTGGGGAGCCAGGCATCCGGGGTGAAGATG
GCCGCCCCGGCCAGGAGGGACCCCGAGGACTCACGGGGCCCCCTGGCAGC
AGGGGAGAGCGTGGGGAGAAGGGTGATGTTGGGAGTGCAGGACTAAAGGG
TGACAAGGGAGACTCAGCTGTGATCCTGGGGCCTCCAGGCCCACGGGGTG
CCAAGGGGGACATGGGTGAACGAGGGCCTCGGGGCTTGGATGGTGACAAA
GGACCTCGGGGAGACAATGGGGACCCTGGTGACAAGGGCAGCAAGGGAGA
GCCTGGTGACAAGGGCTCAGCCGGGTTGCCAGGACTGCGTGGACTCCTGG
GACCCCAGGGTCAACCTGGTGCAGCAGGGATCCCTGGTGACCCGGGATCC
CCAGGAAAGGATGGAGTGCCTGGTATCCGAGGAGAAAAAGGAGATGTTGG
CTTCATGGGTCCCCGGGGCCTCAAGGGTGAACGGGGAGTGAAGGGAGCCT
GTGGCCTTGATGGAGAGAAGGGAGACAAGGGAGAAGCTGGTCCCCCAGGC
CGCCCCGGGCTGGCAGGACACAAAGGAGAGATGGGGGAGCCTGGTGTGCC
GGGCCAGTCGGGGGCCCCTGGCAAGGAGGGCCTGATCGGTCCCAAGGGTG
ACCGAGGCTTTGACGGGCAGCCAGGCCCCAAGGGTGACCAGGGCGAGAAA
GGGGAGCGGGGAACCCCAGGAATTGGGGGCTTCCCAGGCCCCAGTGGAAA
TGATGGCTCTGCTGGTCCCCCAGGGCCACCTGGCAGTGTTGGTCCCAGAG
GCCCCGAAGGACTTCAGGGCCAGAAGGGTGAGCGAGGTCCCCCCGGAGAG
AGAGTGGTGGGGGCTCCTGGGGTCCCTGGAGCTCCTGGCGAGAGAGGGGA
GCAGGGGCGGCCAGGGCCTGCCGGTCCTCGAGGCGAGAAGGGAGAAGCTG
CACTGACGGAGGATGACATCCGGGGCTTTGTGCGCCAAGAGATGAGTCAG
CACTGTGCCTGCCAGGGCCAGTTCATCGCATCTGGATCACGACCCCTCCC
TAGTTATGCTGCAGACACTGCCGGCTCCCAGCTCCATGCTGTGCCTGTGC
TCCGCGTCTCTCATGCAGAGGAGGAAGAGCGGGTACCCCCTGAGGATGAT
GAGTACTCTGAATACTCCGAGTATTCTGTGGAGGAGTACCAGGACCCTGA
AGCTCCTTGGGATAGTGATGACCCCTGTTCCCTGCCACTGGATGAGGGCT
CCTGCACTGCCTACACCCTGCGCTGGTACCATCGGGCTGTGACAGGCAGC
ACAGAGGCCTGTCACCCTTTTGTCTATGGTGGCTGTGGAGGGAATGCCAA
CCGTTTTGGGACCCGTGAGGCCTGCGAGCGCCGCTGCCCACCCCGGGTGG
TCCAGAGCCAGGGGACAGGTACTGCCCAGGACTGA

The protein sequence is:

(SEQ ID NO: 2)
MTLRLLVAALCAGILAEAPRVRAQHRERVTCTRLYAADIVFLLDGSSSIGRSNFREVRSF
LEGLVLPFSGAASAQGVRFATVQYSDDPRTEFGLDALGSGGDVIRAIRELSYKGGNTRTG
AAILHVADHVFLPQLARPGVPKVCILITDGKSQDLVDTAAQRLKGQGVKLFAVGIKNADP
EELKRVASQPTSDFFFFVNDFSILRTLLPLVSRRVCTTAGGVPVTRPPDDSTSAPRDLVL
SEPSSQSLRVQWTAASGPVTGYKVQYTPLTGLGQPLPSERQEVNVPAGETSVRLRGLRPL
TEYQVTVIALYANSIGEAVSGTARTTALEGPELTIQNTTAHSLLVAWRSVPGATGYRVTW
RVLSGGPTQQQELGPGQGSVLLRDLEPGTDYEVTVSTLFGRSVGPATSLMARTDASVEQT
LRPVILGPTSILLSWNLVPEARGYRLEWRRETGLEPPQKVVLPSDVTRYQLDGLQPGTEY
RLTLYTLLEGHEVATPATVVPTGPELPVSPVTDLQATELPGQRVRVSWSPVPGATQYRII
VRSTQGVERTLVLPGSQTAFDLDDVQAGLSYTVRVSARVGPREGSASVLTVRREPETPLA
VPGLRVVVSDATRVRVAWGPVPGASGFRISWSTGSGPESSQTLPPDSTATDITGLQPGTT
YQVAVSVLRGREEGPAAVIVARTDPLGPVRTVHVTQASSSSVTITWTRVPGATGYRVSWH
SAHGPEKSQLVSGEATVAELDGLEPDTEYTVHVRAHVAGVDGPPASVVVRTAPEPVGRVS
RLQILNASSDVLRITWVGVTGATAYRLAWGRSEGGPMRHQILPGNTDSAEIRGLEGGVSY
SVRVTALVGDREGTPVSIVVTTPPEAPPALGTLHVVQRGEHSLRLRWEPVPRAQGFLLHW
QPEGGQEQSRVLGPELSSYHLDGLEPATQYRVRLSVLGPAGEGPSAEVTARTESPRVPSI
ELRVVDTSIDSVTLAWTPVSRASSYILSWRPLRGPGQEVPGSPQTLPGISSSQRVTGLEP
GVSYIFSLTPVLDGVRGPEASVTQTPVCPRGLADVVFLPHATQDNAHRAEATRRVLERLV
LALGPLGPQAVQVGLLSYSHRPSPLFPLNGSHDLGIILQRIRDMPYMDPSGNNLGTAVVT
AHRYMLAPDAPGRRQHVPGVMVLLVDEPLRGDIFSPIREAQASGLNVVMLGMAGADPEQL
RRLAPGMDSVQTFFAVDDGPSLDQAVSGLATALCQASFTTQPRPEPCPVYCPKGQKGEPG
EMGLRGQVGPPGDPGLPGRTGAPGPQGPPGSATAKGERGFPGADGRPGSPGRAGNPGTPG
APGLKGSPGLPGPRGDPGERGPRGPKGEPGAPGQVIGGEGPGLPGRKGDPGPSGPPGPRG
PLGDPGPRGPPGLPGTAMKGDKGDRGERGPPGPGEGGIAPGEPGLPGLPGSPGPQGPVGP
PGKKGEKGDSEDGAPGLPGQPGSPGEQGPRGPPGAIGPKGDRGFPGPLGEAGEKGERGPP
GPAGSRGLPGVAGRPGAKGPEGPPGPTGRQGEKGEPGRPGDPAVVGPAVAGPKGEKGDVG
PAGPRGATGVQGERGPPGLVLPGDPGPKGDPGDRGPIGLTGRAGPPGDSGPPGEKGDPGR
PGPPGPVGPRGRDGEVGEKGDEGPPGDPGLPGKAGERGLRGAPGVRGPVGEKGDQGDPGE
DGRNGSPGSSGPKGDRGEPGPPGPPGRLVDTGPGAREKGEPGDRGQEGPRGPKGDPGLPG
APGERGIEGFRGPPGPQGDPGVRGPAGEKGDRGPPGLDGRSGLDGKPGAAGPSGPNGAAG
KAGDPGRDGLPGLRGEQGLPGPSGPPGLPGKPGEDGKPGLNGKNGEPGDPGEDGRKGEKG
DSGASGREGRDGPKGERGAPGILGPQGPPGLPGPVGPPGQGFPGVPGGTGPKGDRGETGS
KGEQGLPGERGLRGEPGSVPNVDRLLETAGIKASALREIVETWDESSGSFLPVPERRRGP
KGDSGEQGPPGKEGPIGFPGERGLKGDRGDPGPQGPPGLALGERGPPGPSGLAGEPGKPG
IPGLPGRAGGVGEAGRPGERGERGEKGERGEQGRDGPPGLPGTPGPPGPPGPKVSVDEPG
PGLSGEQGPPGLKGAKGEPGSNGDQGPKGDRGVPGIKGDRGEPGPRGQDGNPGLPGERGM
AGPEGKPGLQGPRGPPGPVGGHGDPGPPGAPGLAGPAGPQGPSGLKGEPGETGPPGRGLT
GPTGAVGLPGPPGPSGLVGPQGSPGLPGQVGETGKPGAPGRDGASGKDGDRGSPGVPGSP
GLPGPVGPKGEPGPTGAPGQAVVGLPGAKGEKGAPGGLAGDLVGEPGAKGDRGLPGPRGE
KGEAGRAGEPGDPGEDGQKGAPGPKGFKGDPGVGVPGSPGPPGPPGVKGDLGLPGLPGAP
GVVGFPGQTGPRGEMGQPGPSGERGLAGPPGREGIPGPLGPPGPPGSVGPPGASGLKGDK
GDPGVGLPGPRGERGEPGIRGEDGRPGQEGPRGLTGPPGSRGERGEKGDVGSAGLKGDKG
DSAVILGPPGPRGAKGDMGERGPRGLDGDKGPRGDNGDPGDKGSKGEPGDKGSAGLPGLR
GLLGPQGQPGAAGIPGDPGSPGKDGVPGIRGEKGDVGFMGPRGLKGERGVKGACGLDGEK
GDKGEAGPPGRPGLAGHKGEMGEPGVPGQSGAPGKEGLIGPKGDRGFDGQPGPKGDQGEK
GERGTPGIGGFPGPSGNDGSAGPPGPPGSVGPRGPEGLQGQKGERGPPGERVVGAPGVPG
APGERGEQGRPGPAGPRGEKGEAALTEDDIRGEVRQEMSQHCACQGQFIASGSRPLPSYA
ADTAGSQLHAVPVLRVSHAEEEERVPPEDDEYSEYSEYSVEEYQDPEAPWDSDDPCSLPL
DEGSCTAYTLRWYHRAVTGSTEACHPFVYGGCGGNANRFGTREACERRCPPRVVQSQGTG
TAQD

Preferably, collagen 17 is characterized by the sequence disclosed in the NCBI Data Bank with the Accession no.: NM_000494.3 (COL17A1). The cDNA sequence is:

(SEQ ID NO: 3)
ATGGATGTAACCAAGAAAAACAAACGAGATGGAACTGAAGTCACTGAGAG
AATTGTCACTGAAACAGTAACCACAAGACTTACATCCTTACCACCAAAAG
GCGGGACCAGCAATGGCTATGCTAAAACAGCCTCTCTTGGTGGAGGGAGC
CGGCTGGAGAAACAAAGCCTGACTCATGGCAGCAGCGGCTACATAAACTC
AACTGGAAGCACACGAGGCCATGCCTCCACCTCTAGTTACAGGAGGGCTC
ACTCACCTGCCTCCACTCTGCCCAACTCCCCAGGCTCAACCTTTGAAAGG
AAAACTCACGTTACCCGCCATGCGTATGAAGGGAGCTCCAGTGGCAACTC
TTCTCCGGAGTACCCTCGGAAGGAATTTGCATCTTCTTCAACCAGAGGAC
GGAGTCAAACACGAGAGAGTGAAATTCGAGTTCGACTGCAGAGTGCGTCC
CCATCCACCCGATGGACAGAATTGGATGATGTTAAGCGTTTGCTCAAGGG
GAGTCGATCGGCAAGTGTGAGCCCCACCCGGAATTCCTCCAACACACTCC
CCATCCCCAAGAAAGGCACTGTGGAGACCAAAATTGTGACAGCGAGCTCC
CAGTCGGTGTCAGGCACCTACGATGCAACGATCCTGGATGCCAACCTTCC
CTCCCATGTGTGGTCCTCCACCCTGCCCGCGGGGTCCTCCATGGGGACCT
ATCACAACAACATGACAACCCAGAGCTCATCCCTCCTCAACACCAATGCC
TACTCTGCGGGATCAGTCTTCGGAGTTCCAAACAACATGGCGTCCTGCTC
ACCCACTTTGCACCCTGGACTCAGCACATCCTCCTCAGTGTTTGGCATGC
AGAACAATCTGGCCCCCAGCTTGACCACCCTGTCCCATGGCACCACCACC
ACTTCCACAGCATATGGGGTGAAGAAAAACATGCCCCAGAGTCCTGCGGC
TGTGAACACTGGCGTTTCCACCTCCGCCGCCTGCACCACAAGTGTGCAGA
GCGATGACCTTTTGCACAAGGACTGCAAGTTCCTGATCCTAGAGAAAGAC
AACACACCTGCCAAGAAGGAGATGGAGCTGCTCATCATGACCAAGGACAG
CGGGAAGGTCTTTACAGCCTCCCCTGCCAGCATCGCTGCAACTTCTTTTT
CAGAAGACACCCTAAAAAAAGAAAAGCAAGCTGCCTACAATGCTGACTCA
GGCCTAAAAGCCGAAGCTAATGGAGACCTGAAGACTGTGTCCACAAAGGG
CAAGACCACCACTGCAGATATCCACAGCTACGGCAGCAGTGGTGGTGGTG
GCAGTGGAGGAGGTGGCGGTGTTGGTGGCGCTGGCGGCGGCCCTTGGGGA
CCAGCGCCAGCCTGGTGCCCCTGCGGCTCCTGCTGCAGCTGGTGGAAGTG
GCTGCTGGGCCTGCTGCTCACCTGGCTGCTACTCCTGGGGCTGCTCTTCG
GCCTCATTGCTCTGGCGGAGGAGGTGAGGAAGCTGAAGGCGCGTGTGGAT
GAGCTGGAGAGGATCAGGAGGAGCATACTGCCCTATGGGGACAGCATGGA
TAGAATAGAAAAGGACCGCCTCCAGGGCATGGCACCCGCGGCGGGAGCAG
ACCTGGACAAAATTGGGCTGCACAGTGACAGCCAGGAGGAGCTCTGGATG
TTCGTGAGGAAGAAGCTAATGATGGAACAGGAAAATGGAAATCTCCGAGG
AAGCCCTGGCCCTAAAGGTGACATGGGAAGTCCAGGCCCTAAAGGAGATC
GAGGGTTCCCTGGGACTCCAGGTATCCCTGGGCCCTTGGGCCACCCAGGT
CCACAAGGACCAAAGGGTCAAAAAGGCAGCGTGGGAGATCCTGGCATGGA
AGGCCCCATGGGCCAGAGAGGGCGAGAAGGCCCCATGGGACCTCGTGGTG
AGGCAGGGCCTCCTGGATCTGGAGAGAAAGGGGAAAGAGGGGCTGCTGGT
GAACCAGGTCCTCATGGCCCACCTGGTGTCCCAGGTTCTGTGGGTCCCAA
AGGTTCCAGCGGCTCTCCTGGCCCACAGGGCCCTCCAGGTCCTGTAGGTC
TCCAAGGGCTCCGAGGTGAAGTAGGACTTCCTGGTGTCAAAGGTGACAAA
GGACCAATGGGACCACCAGGACCCAAAGGTGACCAGGGTGAGAAAGGACC
TCGAGGCCTCACAGGCGAGCCTGGCATGAGAGGTTTGCCTGGTGCTGTTG
GTGAGCCCGGGGCTAAAGGAGCAATGGGTCCTGCTGGCCCAGACGGACAC
CAAGGCCCAAGAGGTGAACAAGGTCTTACTGGGATGCCTGGAATCCGTGG
CCCACCAGGACCTTCTGGAGACCCAGGAAAGCCAGGTCTCACAGGACCCC
AGGGACCTCAGGGACTTCCCGGTACCCCTGGCCGACCAGGAATAAAAGGT
GAACCAGGAGCTCCAGGCAAGATCGTGACTTCGGAGGGGTCATCGATGCT
CACTGTCCCAGGCCCCCCAGGACCTCCTGGAGCCATGGGACCCCCAGGAC
CTCCAGGTGCCCCAGGCCCTGCCGGCCCAGCTGGTCTCCCAGGACATCAA
GAAGTTCTTAATTTACAAGGTCCCCCAGGCCCACCCGGCCCACGCGGGCC
ACCAGGGCCTTCCATTCCAGGCCCACCAGGACCCCGAGGCCCACCAGGGG
AGGGTTTGCCAGGCCCACCAGGCCCACCAGGATCGTTCCTGTCCAACTCA
GAAACCTTCCTCTCCGGCCCCCCAGGCCCACCTGGCCCCCCAGGTCCCAA
GGGAGACCAAGGTCCCCCAGGCCCCAGAGGACACCAAGGCGAGCAAGGCC
TCCCAGGTTTCTCAACCTCAGGGTCCAGTTCTTTCGGACTCAACCTTCAG
GGACCACCAGGCCCACCTGGCCCCCAGGGACCCAAAGGTGACAAAGGTGA
TCCAGGTGTTCCAGGGGCTCTTGGCATTCCTAGTGGTCCTTCTGAAGGGG
GATCATCAAGTACCATGTACGTGTCAGGCCCGCCAGGGCCCCCTGGGCCC
CCTGGGCCTCCGGGCTCTATCAGCAGCTCTGGCCAGGAGATTCAGCAGTA
CATCTCTGAGTACATGCAGAGTGACAGTATTAGATCTTACCTATCCGGAG
TTCAGGGTCCCCCAGGCCCACCTGGTCCCCCAGGACCTGTCACCACCATC
ACAGGCGAGACTTTCGACTACTCAGAGCTGGCAAGCCACGTTGTGAGCTA
CTTACGGACTTCGGGGTACGGTGTCAGCTTGTTCTCGTCCTCCATCTCTT
CTGAAGACATTCTGGCTGTGCTGCAGCGGGATGACGTGCGTCAGTACCTA
CGTCAGTACTTGATGGGCCCTCGGGGTCCGCCAGGGCCACCAGGAGCCAG
TGGAGATGGGTCCCTCCTGTCTTTGGACTATGCAGAGCTGAGTAGTCGCA
TTCTCAGCTACATGTCGAGTTCTGGGATCAGCATTGGGCTTCCTGGTCCC
CCGGGGCCCCCTGGCTTGCCGGGAACCTCCTATGAGGAGCTCCTCTCCTT
GCTGCGAGGGTCTGAATTCAGAGGCATCGTTGGACCCCCAGGTCCCCCGG
GTCCACCAGGGATCCCAGGCAATGTGTGGTCCAGCATCAGCGTGGAGGAC
CTCTCGTCTTACTTACATACTGCCGGCTTGTCATTCATCCCAGGCCCTCC
AGGACCTCCTGGTCCCCCAGGGCCTCGAGGGCCCCCGGGTGTCTCAGGAG
CCCTGGCAACCTATGCAGCTGAAAACAGCGACAGCTTCCGGAGCGAGCTG
ATCAGCTACCTCACAAGTCCTGATGTGCGCAGCTTCATTGTTGGCCCCCC
AGGCCCTCCTGGGCCGCAGGGACCCCCTGGGGACAGCCGCCTCCTGTCCA
CGGATGCCTCCCACAGTCGGGGTAGCAGCTCCTCCTCACACAGCTCATCT
GTCAGGCGGGGCAGCTCCTACAGCTCTTCCATGAGCACAGGAGGAGGTGG
TGCAGGCTCCCTGGGTGCAGGCGGTGCCTTTGGTGAAGCTGCAGGAGACA
GGGGTCCCTATGGCACTGACATCGGCCCAGGCGGAGGCTATGGGGCAGCA
GCAGAAGGCGGCATGTATGCTGGCAATGGCGGACTATTGGGAGCTGACTT
TGCTGGAGATCTGGATTACAATGAGCTGGCTGTGAGGGTGTCAGAGAGCA
TGCAGCGTCAGGGCCTACTGCAAGGGATGGCCTACACTGTCCAGGGCCCA
CCAGGCCAGCCTGGGCCACAGGGGCCACCCGGCATCAGCAAGGTCTTCTC
TGCCTACAGCAACGTGACTGCGGACCTCATGGACTTCTTCCAAACTTATG
GAGCCATTCAAGGACCCCCTGGGCAAAAAGGAGAGATGGGCACTCCAGGA
CCCAAAGGTGACAGGGGCCCTGCTGGGCCACCAGGTCATCCTGGGCCACC
TGGCCCTCGAGGACACAAGGGAGAAAAAGGAGACAAAGGTGACCAAGTCT
ATGCTGGGCGGAGAAGGAGAAGAAGTATTGCTGTCAAGCCGTGA

The protein sequence is:

(SEQ ID NO: 4)
MDVTKKNKRDGTEVTERIVTETVTTRLTSLPPKGGTSNGYAKTASLGGGSRLEKQSLTHG
SSGYINSTGSTRGHASTSSYRRAHSPASTLPNSPGSTFERKTHVTRHAYEGSSSGNSSPE
YPRKEFASSSTRGRSQTRESEIRVRLQSASPSTRWTELDDVKRLLKGSRSASVSPTRNSS
NTLPIPKKGTVETKIVTASSQSVSGTYDATILDANLPSHVWSSTLPAGSSMGTYHNNMTT
QSSSLLNTNAYSAGSVFGVPNNMASCSPTLHPGLSTSSSVFGMQNNLAPSLTTLSHGTTT
TSTAYGVKKNMPQSPAAVNTGVSTSAACTTSVQSDDLLHKDCKFLILEKDNTPAKKEMEL
LIMTKDSGKVFTASPASIAATSFSEDTLKKEKQAAYNADSGLKAEANGDLKTVSTKGKTT
TADIHSYGSSGGGGSGGGGGVGGAGGGPWGPAPAWCPCGSCCSWWKWLLGLLLTWLLLLG
LLFGLIALAEEVRKLKARVDELERIRRSILPYGDSMDRIEKDRLQGMAPAAGADLDKIGL
HSDSQEELWMFVRKKLMMEQENGNLRGSPGPKGDMGSPGPKGDRGFPGTPGIPGPLGHPG
PQGPKGQKGSVGDPGMEGPMGQRGREGPMGPRGEAGPPGSGEKGERGAAGEPGPHGPPGV
PGSVGPKGSSGSPGPQGPPGPVGLQGLRGEVGLPGVKGDKGPMGPPGPKGDQGEKGPRGL
TGEPGMRGLPGAVGEPGAKGAMGPAGPDGHQGPRGEQGLTGMPGIRGPPGPSGDPGKPGL
TGPQGPQGLPGTPGRPGIKGEPGAPGKIVTSEGSSMLTVPGPPGPPGAMGPPGPPGAPGP
AGPAGLPGHQEVLNLQGPPGPPGPRGPPGPSIPGPPGPRGPPGEGLPGPPGPPGSFLSNS
ETFLSGPPGPPGPPGPKGDQGPPGPRGHQGEQGLPGFSTSGSSSFGLNLQGPPGPPGPQG
PKGDKGDPGVPGALGIPSGPSEGGSSSTMYVSGPPGPPGPPGPPGSISSSGQEIQQYISE
YMQSDSIRSYLSGVQGPPGPPGPPGPVTTITGETFDYSELASHVVSYLRTSGYGVSLFSS
SISSEDILAVLQRDDVRQYLRQYLMGPRGPPGPPGASGDGSLLSLDYAELSSRILSYMSS
SGISIGLPGPPGPPGLPGTSYEELLSLLRGSEFRGIVGPPGPPGPPGIPGNVWSSISVED
LSSYLHTAGLSFIPGPPGPPGPPGPRGPPGVSGALATYAAENSDSFRSELISYLTSPDVR
SFIVGPPGPPGPQGPPGDSRLLSTDASHSRGSSSSSHSSSVRRGSSYSSSMSTGGGGAGS
LGAGGAFGEAAGDRGPYGTDIGPGGGYGAAAEGGMYAGNGGLLGADFAGDLDYNELAVRV
SESMQRQGLLQGMAYTVQGPPGQPGPQGPPGISKVFSAYSNVTADLMDFFQTYGAIQGPP
GQKGEMGTPGPKGDRGPAGPPGHPGPPGPRGHKGEKGDKGDQVYAGRRRRRSIAVKP

Preferably, the beta-3 chain of laminin 5 is characterized by the sequence disclosed in the NCBI Data Bank with the Accession no.: NM_000228-Q13751 (LAMB3). The cDNA sequence is:

(SEQ ID NO: 5)
ATGAGACCATTCTTCCTCTTGTGTTTTGCCCTGCCTGGCCTCCTGCATGC
CCAACAAGCCTGCTCCCGTGGGGCCTGCTATCCACCTGTTGGGGACCTGC
TTGTTGGGAGGACCCGGTTTCTCCGAGCTTCATCTACCTGTGGACTGACC
AAGCCTGAGACCTACTGCACCCAGTATGGCGAGTGGCAGATGAAATGCTG
CAAGTGTGACTCCAGGCAGCCTCACAACTACTACAGTCACCGAGTAGAGA
ATGTGGCTTCATCCTCCGGCCCCATGCGCTGGTGGCAGTCACAGAATGAT
GTGAACCCTGTCTCTCTGCAGCTGGACCTGGACAGGAGATTCCAGCTTCA
AGAAGTCATGATGGAGTTCCAGGGGCCCATGCCCGCCGGCATGCTGATTG
AGCGCTCCTCAGACTTCGGTAAGACCTGGCGAGTGTACCAGTACCTGGCT
GCCGACTGCACCTCCACCTTCCCTCGGGTCCGCCAGGGTCGGCCTCAGAG
CTGGCAGGATGTTCGGTGCCAGTCCCTGCCTCAGAGGCCTAATGCACGCC
TAAATGGGGGGAAGGTCCAACTTAACCTTATGGATTTAGTGTCTGGGATT
CCAGCAACTCAAAGTCAAAAAATTCAAGAGGTGGGGGAGATCACAAACTT
GAGAGTCAATTTCACCAGGCTGGCCCCTGTGCCCCAAAGGGGCTACCACC
CTCCCAGCGCCTACTATGCTGTGTCCCAGCTCCGTCTGCAGGGGAGCTGC
TTCTGTCACGGCCATGCTGATCGCTGCGCACCCAAGCCTGGGGCCTCTGC
AGGCCCCTCCACCGCTGTGCAGGTCCACGATGTCTGTGTCTGCCAGCACA
ACACTGCCGGCCCAAATTGTGAGCGCTGTGCACCCTTCTACAACAACCGG
CCCTGGAGACCGGCGGAGGGCCAGGACGCCCATGAATGCCAAAGGTGCGA
CTGCAATGGGCACTCAGAGACATGTCACTTTGACCCCGCTGTGTTTGCCG
CCAGCCAGGGGGCATATGGAGGTGTGTGTGACAATTGCCGGGACCACACC
GAAGGCAAGAACTGTGAGCGGTGTCAGCTGCACTATTTCCGGAACCGGCG
CCCGGGAGCTTCCATTCAGGAGACCTGCATCTCCTGCGAGTGTGATCCGG
ATGGGGCAGTGCCAGGGGCTCCCTGTGACCCAGTGACCGGGCAGTGTGTG
TGCAAGGAGCATGTGCAGGGAGAGCGCTGTGACCTATGCAAGCCGGGCTT
CACTGGACTCACCTACGCCAACCCGCAGGGCTGCCACCGCTGTGACTGCA
ACATCCTGGGGTCCCGGAGGGACATGCCGTGTGACGAGGAGAGTGGGCGC
TGCCTTTGTCTGCCCAACGTGGTGGGTCCCAAATGTGACCAGTGTGCTCC
CTACCACTGGAAGCTGGCCAGTGGCCAGGGCTGTGAACCGTGTGCCTGCG
ACCCGCACAACTCCCTCAGCCCACAGTGCAACCAGTTCACAGGGCAGTGC
CCCTGTCGGGAAGGCTTTGGTGGCCTGATGTGCAGCGCTGCAGCCATCCG
CCAGTGTCCAGACCGGACCTATGGAGACGTGGCCACAGGATGCCGAGCCT
GTGACTGTGATTTCCGGGGAACAGAGGGCCCGGGCTGCGACAAGGCATCA
GGCCGCTGCCTCTGCCGCCCTGGCTTGACCGGGCCCCGCTGTGACCAGTG
CCAGCGAGGCTACTGTAATCGCTACCCGGTGTGCGTGGCCTGCCACCCTT
GCTTCCAGACCTATGATGCGGACCTCCGGGAGCAGGCCCTGCGCTTTGGT
AGACTCCGCAATGCCACCGCCAGCCTGTGGTCAGGGCCTGGGCTGGAGGA
CCGTGGCCTGGCCTCCCGGATCCTAGATGCAAAGAGTAAGATTGAGCAGA
TCCGAGCAGTTCTCAGCAGCCCCGCAGTCACAGAGCAGGAGGTGGCTCAG
GTGGCCAGTGCCATCCTCTCCCTCAGGCGAACTCTCCAGGGCCTGCAGCT
GGATCTGCCCCTGGAGGAGGAGACGTTGTCCCTTCCGAGAGACCTGGAGA
GTCTTGACAGAAGCTTCAATGGTCTCCTTACTATGTATCAGAGGAAGAGG
GAGCAGTTTGAAAAAATAAGCAGTGCTGATCCTTCAGGAGCCTTCCGGAT
GCTGAGCACAGCCTACGAGCAGTCAGCCCAGGCTGCTCAGCAGGTCTCCG
ACAGCTCGCGCCTTTTGGACCAGCTCAGGGACAGCCGGAGAGAGGCAGAG
AGGCTGGTGCGGCAGGCGGGAGGAGGAGGAGGCACCGGCAGCCCCAAGCT
TGTGGCCCTGAGGCTGGAGATGTCTTCGTTGCCTGACCTGACACCCACCT
TCAACAAGCTCTGTGGCAACTCCAGGCAGATGGCTTGCACCCCAATATCA
TGCCCTGGTGAGCTATGTCCCCAAGACAATGGCACAGCCTGTGGCTCCCG
CTGCAGGGGTGTCCTTCCCAGGGCCGGTGGGGCCTTCTTGATGGCGGGGC
AGGTGGCTGAGCAGCTGCGGGGCTTCAATGCCCAGCTCCAGCGGACCAGG
CAGATGATTAGGGCAGCCGAGGAATCTGCCTCACAGATTCAATCCAGTGC
CCAGCGCTTGGAGACCCAGGTGAGCGCCAGCCGCTCCCAGATGGAGGAAG
ATGTCAGACGCACACGGCTCCTAATCCAGCAGGTCCGGGACTTCCTAACA
GACCCCGACACTGATGCAGCCACTATCCAGGAGGTCAGCGAGGCCGTGCT
GGCCCTGTGGCTGCCCACAGACTCAGCTACTGTTCTGCAGAAGATGAATG
AGATCCAGGCCATTGCAGCCAGGCTCCCCAACGTGGACTTGGTGCTGTCC
CAGACCAAGCAGGACATTGCGCGTGCCCGCCGGTTGCAGGCTGAGGCTGA
GGAAGCCAGGAGCCGAGCCCATGCAGTGGAGGGCCAGGTGGAAGATGTGG
TTGGGAACCTGCGGCAGGGGACAGTGGCACTGCAGGAAGCTCAGGACACC
ATGCAAGGCACCAGCCGCTCCCTTCGGCTTATCCAGGACAGGGTTGCTGA
GGTTCAGCAGGTACTGCGGCCAGCAGAAAAGCTGGTGACAAGCATGACCA
AGCAGCTGGGTGACTTCTGGACACGGATGGAGGAGCTCCGCCACCAAGCC
CGGCAGCAGGGGGCAGAGGCAGTCCAGGCCCAGCAGCTTGCGGAAGGTGC
CAGCGAGCAGGCATTGAGTGCCCAAGAGGGATTTGAGAGAATAAAACAAA
AGTATGCTGAGTTGAAGGACCGGTTGGGTCAGAGTTCCATGCTGGGTGAG
CAGGGTGCCCGGATCCAGAGTGTGAAGACAGAGGCAGAGGAGCTGTTTGG
GGAGACCATGGAGATGATGGACAGGATGAAAGACATGGAGTTGGAGCTGC
TGCGGGGCAGCCAGGCCATCATGCTGCGCTCAGCGGACCTGACAGGACTG
GAGAAGCGTGTGGAGCAGATCCGTGACCACATCAATGGGCGCGTGCTCTA
CTATGCCACCTGCAAGTGA

The protein sequence is:

(SEQ ID NO: 6)
MRPFFLLCFALPGLLHAQQACSRGACYPPVGDLLVGRTRFLRASSTCGLT
KPETYCTQYGEWQMKCCKCDSRQPHNYYSHRVENVASSSGPMRWWQSQND
VNPVSLQLDLDRRFQLQEVMMEFQGPMPAGMLIERSSDFGKTWRVYQYLA
ADCTSTFPRVRQGRPQSWQDVRCQSLPQRPNARLNGGKVQLNLMDLVSGI
PATQSQKIQEVGEITNLRVNFTRLAPVPQRGYHPPSAYYAVSQLRLQGSC
FCHGHADRCAPKPGASAGPSTAVQVHDVCVCQHNTAGPNCERCAPFYNNR
PWRPAEGQDAHECQRCDCNGHSETCHFDPAVFAASQGAYGGVCDNCRDHT
EGKNCERCQLHYFRNRRPGASIQETCISCECDPDGAVPGAPCDPVTGQCV
CKEHVQGERCDLCKPGFTGLTYANPQGCHRCDCNILGSRRDMPCDEESGR
CLCLPNVVGPKCDQCAPYHWKLASGQGCEPCACDPHNSLSPQCNQFTGQC
PCREGFGGLMCSAAAIRQCPDRTYGDVATGCRACDCDFRGTEGPGCDKAS
GRCLCRPGLTGPRCDQCQRGYCNRYPVCVACHPCFQTYDADLREQALRFG
RLRNATASLWSGPGLEDRGLASRILDAKSKIEQIRAVLSSPAVTEQEVAQ
VASAILSLRRTLQGLQLDLPLEEETLSLPRDLESLDRSFNGLLTMYQRKR
EQFEKISSADPSGAFRMLSTAYEQSAQAAQQVSDSSRLLDQLRDSRREAE
RLVRQAGGGGGTGSPKLVALRLEMSSLPDLTPTFNKLCGNSRQMACTPIS
CPGELCPQDNGTACGSRCRGVLPRAGGAFLMAGQVAEQLRGFNAQLQRTR
QMIRAAEESASQIQSSAQRLETQVSASRSQMEEDVRRTRLLIQQVRDFLT
DPDTDAATIQEVSEAVLALWLPTDSATVLQKMNEIQAIAARLPNVDLVLS
QTKQDIARARRLQAEAEEARSRAHAVEGQVEDVVGNLRQGTVALQEAQDT
MQGTSRSLRLIQDRVAEVQQVLRPAEKLVTSMTKQLGDFWTRMEELRHQA
RQQGAEAVQAQQLAEGASEQALSAQEGFERIKQKYAELKDRLGQSSMLGE
QGARIQSVKTEAEELFGETMEMMDRMKDMELELLRGSQAIMLRSADLTGL
EKRVEQIRDHINGRVLYYATCK

Preferably, the LAMA3 (α3 chain of laminin 5) is characterized by the sequence disclosed in the NCBI Data Bank with the Accession no.: NP_937762.1. Its cDNA sequence is:

(SEQ ID NO: 7)
ATGGCGGCGGCCGCGCGGCCTCGGGGTCGGGCACTGGGGCCAGTACTGCC
GCCGACGCCGCTGCTCCTGCTGGTACTGCGGGTGCTGCCAGCCTGCGGGG
CGACCGCTCGGGATCCCGGGGCCGCGGCCGGGCTCAGCCTTCACCCGACT
TACTTCAACCTGGCCGAGGCGGCGAGGATTTGGGCCACCGCCACCTGCGG
GGAGAGGGGACCCGGCGAGGGGAGGCCCCAGCCCGAGCTCTACTGCAAGT
TGGTCGGGGGCCCCACCGCCCCAGGCAGCGGCCACACCATCCAGGGCCAG
TTCTGTGACTATTGCAATTCTGAAGACCCCAGGAAAGCACATCCTGTCAC
CAATGCCATCGATGGATCTGAACGTTGGTGGCAAAGCCCTCCCCTGTCCT
CAGGCACACAGTACAACAGAGTCAACCTCACCTTGGATCTGGGGCAGCTC
TTCCATGTGGCCTATATTTTAATCAAATTTGCAAATTCTCCTCGCCCTGA
TCTTTGGGTCTTGGAAAGATCTGTAGACTTTGGAAGCACCTACTCACCAT
GGCAATATTTTGCTCATTCTAAAGTAGACTGTTTAAAAGAATTTGGGCGG
GAGGCAAATATGGCTGTCACCCGGGATGATGATGTACTTTGTGTTACTGA
ATATTCCCGTATTGTACCTTTGGAAAATGGTGAGGTTGTGGTGTCCTTGA
TAAACGGTCGTCCAGGTGCAAAAAATTTTACTTTCTCTCACACCCTGAGG
GAGTTTACCAAGGCAACAAACATCCGCTTGCGTTTTCTTAGAACCAATAC
GCTTCTTGGACACCTCATCTCCAAAGCCCAGCGAGATCCAACTGTCACTC
GGCGGTATTATTACAGCATAAAGGACATCAGCATTGGTGGGCAGTGTGTT
TGCAATGGCCATGCTGAAGTGTGCAATATAAACAATCCTGAAAAACTGTT
TCGGTGTGAATGCCAGCACCACACCTGTGGGGAGACGTGTGATCGCTGCT
GCACAGGGTACAATCAGAGGCGCTGGCGGCCCGCCGCTTGGGAGCAGAGC
CACGAGTGTGAAGCATGCAACTGCCACGGCCATGCCAGCAACTGTTACTA
TGATCCAGATGTTGAGCGGCAGCAGGCAAGCTTGAATACCCAGGGCATCT
ATGCTGGTGGAGGGGTCTGCATTAACTGTCAGCACAACACAGCTGGAGTA
AACTGTGAACAGTGTGCTAAGGGCTATTACCGCCCTTATGGGGTTCCAGT
GGATGCCCCTGATGGCTGCATCCCCTGCAGCTGTGACCCTGAGCATGCGG
ATGGCTGTGAACAGGGTTCAGGCCGCTGTCACTGCAAGCCAAATTTCCAC
GGAGACAACTGTGAGAAGTGTGCAATTGGATACTACAATTTCCCATTTTG
CTTGAGAATTCCCATTTTTCCTGTTTCTACACCAAGTTCAGAAGATCCAG
TAGCTGGAGATATAAAAGGGTGTGACTGTAATCTGGAAGGTGTTCTCCCT
GAAATATGTGATGCCCACGGACGGTGCCTGTGCCGCCCTGGGGTTGAGGG
CCCTCGATGTGATACCTGCCGCTCTGGTTTCTACTCATTCCCTATTTGCC
AAGCCTGCTGGTGTTCAGCCCTTGGATCCTACCAGATGCCCTGCAGCTCA
GTGACTGGACAGTGTGAATGTCGGCCAGGAGTTACAGGACAGCGGTGTGA
CAGGTGTCTCTCAGGAGCTTATGATTTCCCCCACTGCCAAGGTTCCAGCA
GTGCTTGTGACCCAGCTGGTACCATCAACTCCAATTTGGGGTATTGCCAA
TGCAAGCTTCATGTTGAAGGTCCTACTTGTAGCCGCTGCAAACTGTTATA
TTGGAATCTGGACAAAGAAAACCCCAGTGGATGTTCAGAATGCAAGTGCC
ATAAGGCGGGAACAGTGAGTGGAACTGGAGAGTGTAGGCAGGGAGATGGT
GACTGTCACTGCAAGTCCCATGTGGGTGGCGATTCCTGCGACACCTGTGA
AGATGGATATTTTGCTTTGGAAAAGAGCAATTACTTTGGGTGTCAAGGGT
GTCAGTGTGACATTGGTGGGGCATTGTCCTCCATGTGCAGTGGGCCCTCG
GGAGTGTGCCAGTGCCGAGAGCATGTCGTGGGAAAGGTGTGCCAGCGGCC
TGAAAACAACTACTATTTCCCAGATTTGCATCATATGAAGTATGAGATTG
AAGACGGCAGCACACCTAATGGGAGAGACCTTCGATTTGGATTTGATCCG
CTGGCATTTCCTGAGTTTAGCTGGAGAGGATATGCCCAAATGACCTCAGT
ACAGAATGATGTAAGAATAACATTGAATGTAGGGAAGTCAAGTGGCTCCT
TGTTTCGTGTTATTCTGAGATACGTTAACCCTGGAACTGAAGCAGTATCT
GGCCATATAACTATTTATCCATCCTGGGGTGCTGCTCAAAGCAAAGAGAT
CATCTTCCTGCCGAGTAAGGAGCCAGCCTTTGTCACTGTCCCTGGAAATG
GTTTTGCAGACCCATTTTCAATCACACCAGGAATATGGGTTGCTTGTATT
AAGGCAGAAGGAGTCCTTCTGGATTACCTGGTGCTGCTCCCCAGGGACTA
CTATGAAGCCTCTGTACTGCAGCTGCCAGTCACAGAACCATGTGCCTACG
CAGGACCTCCCCAAGAAAATTGCTTACTCTACCAGCATTTGCCAGTGACC
AGATTCCCCTGTACCCTGGCTTGTGAGGCCAGACACTTCCTGCTTGATGG
GGAGCCAAGACCCGTGGCAGTGAGGCAGCCCACACCTGCACACCCTGTCA
TGGTGGACCTCAGCGGGAGAGAGGTGGAATTGCATCTGCGGCTGCGCATC
CCACAGGTTGGCCACTACGTGGTTGTGGTCGAGTATTCCACGGAGGCAGC
TCAGCTGTTTGTGGTTGATGTGAATGTGAAGAGCTCCGGGTCTGTTCTGG
CAGGCCAGGTGAACATTTACAGCTGCAACTACAGTGTTCTCTGCCGGAGT
GCTGTGATTGATCACATGAGCCGCATCGCCATGTATGAGCTATTGGCAGA
TGCAGACATTCAGCTCAAGGGACACATGGCCCGATTCCTTCTGCATCAAG
TTTGTATCATACCTATTGAAGAATTCTCAGCTGAGTATGTGAGACCACAA
GTCCACTGCATTGCCAGTTATGGGCGATTTGTCAATCAAAGTGCCACCTG
TGTCTCCTTGGCCCATGAAACTCCTCCAACAGCATTAATTTTGGATGTTC
TAAGTGGCAGGCCTTTCCCTCACCTGCCCCAGCAGTCGTCACCTTCTGTT
GATGTTCTTCCTGGGGTCACCTTGAAGGCACCGCAGAATCAAGTGACCCT
GAGAGGACGTGTACCACACCTGGGCCGATACGTCTTTGTCATCCATTTTT
ACCAAGCAGCGCACCCGACGTTTCCCGCGCAGGTGTCGGTGGATGGCGGG
TGGCCACGGGCAGGCTCCTTCCATGCCTCTTTTTGCCCCCATGTGCTTGG
CTGCCGGGATCAAGTGATTGCCGAAGGCCAGATTGAGTTTGACATCTCAG
AGCCTGAAGTGGCCGCAACTGTGAAGGTTCCAGAAGGAAAGTCCTTGGTT
TTGGTCCGTGTTCTAGTGGTGCCTGCAGAAAACTATGACTACCAAATACT
TCACAAAAAATCCATGGACAAGTCACTCGAGTTTATCACCAATTGTGGAA
AAAACAGCTTTTACCTTGACCCCCAGACAGCCTCCAGATTCTGTAAGAAT
TCCGCCAGGTCCCTGGTGGCCTTTTACCACAAGGGCGCCCTGCCTTGTGA
GTGCCACCCCACTGGGGCCACCGGCCCTCACTGCAGCCCTGAGGGTGGGC
AGTGCCCATGCCAGCCCAACGTCATCGGGCGGCAGTGCACCCGCTGTGCA
ACAGGCCACTACGGATTCCCACGCTGCAAGCCGTGCAGCTGTGGTCGGCG
CCTTTGTGAAGAGATGACGGGGCAGTGCCGCTGCCCTCCCCGCACGGTCA
GGCCCCAGTGTGAGGTGTGTGAGACACACTCATTCAGCTTCCACCCCATG
GCCGGCTGCGAAGGCTGCAACTGTTCCAGGAGGGGCACCATCGAGGCTGC
CATGCCGGAGTGTGACCGGGACAGCGGGCAGTGCAGATGCAAGCCCAGAA
TCACAGGGCGGCAGTGTGACCGATGTGCTTCCGGGTTTTACCGCTTTCCT
GAGTGTGTTCCCTGCAATTGCAACAGAGATGGGACTGAGCCAGGAGTGTG
TGACCCAGGGACCGGGGCTTGCCTCTGCAAGGAAAATGTAGAAGGCACAG
AGTGTAATGTGTGTCGAGAAGGCTCATTCCATTTGGACCCAGCCAATCTC
AAGGGTTGTACCAGCTGTTTCTGTTTTGGAGTAAATAATCAATGTCACAG
CTCACATAAGCGAAGGACTAAGTTTGTGGATATGCTGGGCTGGCACCTGG
AGACAGCAGACAGAGTGGACATCCCTGTCTCTTTCAACCCAGGCAGCAAC
AGTATGGTGGCGGATCTCCAGGAGCTGCCCGCAACCATCCACAGCGCGTC
CTGGGTCGCACCCACCTCCTACCTGGGGGACAAGGTTTCTTCATATGGTG
GTTACCTCACTTACCAAGCCAAGTCCTTTGGCTTGCCTGGCGACATGGTT
CTTCTGGAAAAGAAGCCGGATGTACAGCTCACTGGTCAGCACATGTCCAT
CATCTATGAGGAGACAAACACCCCACGGCCAGACCGGCTGCATCATGGAC
GAGTGCACGTGGTCGAGGGAAACTTCAGACATGCCAGCAGCCGTGCCCCA
GTGTCTAGGGAGGAGCTGATGACAGTGCTGTCTAGACTGGCAGATGTGCG
CATCCAAGGCCTCTACTTCACAGAGACTCAAAGGCTCACCCTGAGCGAGG
TGGGGCTAGAGGAAGCCTCTGACACAGGAAGTGGGCGCATAGCACTTGCT
GTGGAAATCTGTGCCTGCCCCCCTGCCTACGCTGGTGACTCTTGTCAGGG
TTGTAGCCCTGGATACTATCGGGATCATAAAGGCTTGTATACCGGACGGT
GTGTTCCCTGCAATTGCAACGGACATTCAAATCAATGCCAGGATGGCTCA
GGCATATGTGTTAACTGTCAGCACAACACCGCGGGAGAGCACTGTGAACG
CTGCCAGGAGGGCTACTATGGCAACGCCGTCCACGGATCCTGCAGGGCCT
GCCCATGTCCTCACACTAACAGCTTTGCCACTGGCTGTGTGGTGAATGGG
GGAGACGTGCGGTGCTCCTGCAAAGCTGGGTACACAGGAACACAGTGTGA
AAGGTGTGCACCGGGATATTTCGGGAATCCCCAGAAATTCGGAGGTAGCT
GCCAACCATGCAGTTGTAACAGCAATGGCCAGCTGGGCAGCTGTCATCCC
CTGACTGGAGACTGCATAAACCAAGAACCCAAAGATAGCAGCCCTGCAGA
AGAATGTGATGATTGCGACAGCTGTGTGATGACCCTCCTGAACGACCTGG
CCACCATGGGCGAGCAGCTCCGCCTGGTCAAGTCTCAGCTGCAGGGCCTG
AGTGCCAGCGCAGGGCTTCTGGAGCAGATGAGGCACATGGAGACCCAGGC
CAAGGACCTGAGGAATCAGTTGCTCAACTACCGTTCTGCCATTTCAAATC
ATGGATCAAAAATAGAAGGCCTGGAAAGAGAACTGACTGATTTGAATCAA
GAATTTGAGACTTTGCAAGAAAAGGCTCAAGTAAATTCCAGAAAAGCACA
AACATTAAACAACAATGTTAATCGGGCAACACAAAGCGCAAAAGAACTGG
ATGTGAAGATTAAAAATGTCATCCGGAATGTGCACATGCTGAACCGGATA
AGGACCTGGCAGAAAACCCACCAGGGGGAGAACAATGGGCTTGCTAACAG
TATCCGGGATTCTTTAAATGAATACGAAGCCAAACTCAGTGACCTTCGTG
CTCGGCTGCAGGAGGCAGCTGCCCAAGCCAAGCAGGCAAATGGCTTGAAC
CAAGAAAACGAGAGAGCTTTGGGAGCCATTCAGAGACAAGTGAAAGAAAT
AAATTCCCTGCAGAGTGATTTCACCAAGTATCTAACCACTGCAGACTCAT
CTTTGTTGCAAACCAACATTGCGCTGCAGCTGATGGAGAAAAGCCAGAAG
GAATATGAAAAATTAGCTGCCAGTTTAAATGAAGCAAGACAAGAACTAAG
TGACAAAGTAAGAGAACTTTCCAGATCTGCTGGCAAAACATCCCTTGTGG
AGGAGGCAGAAAAGCACGCGCGGTCCTTACAAGAGCTGGCAAAGCAGCTG
GAAGAGATCAAGAGAAACGCCAGCGGGGATGAGCTGGTGCGCTGTGCTGT
GGATGCCGCCACCGCCTACGAGAACATCCTCAATGCCATCAAAGCGGCCG
AGGACGCAGCCAACAGGGCTGCCAGTGCATCTGAATCTGCCCTCCAGACA
GTGATAAAGGAAGATCTGCCAAGAAAAGCTAAAACCCTGAGTTCCAACAG
TGATAAACTGTTAAATGAAGCCAAGATGACACAAAAGAAGCTAAAGCAAG
AAGTCAGTCCAGCTCTCAACAACCTACAGCAAACCCTGAATATTGTGACA
GTTCAGAAAGAAGTGATAGACACCAATCTCACAACTCTCCGAGATGGTCT
TCATGGGATACAGAGAGGTGATATTGATGCTATGATCAGTAGTGCAAAGA
GCATGGTCAGAAAGGCCAACGACATCACAGATGAGGTTCTGGATGGGCTC
AACCCCATCCAGACAGATGTGGAAAGAATTAAGGACACCTATGGGAGGAC
ACAGAACGAAGACTTCAAAAAGGCTCTGACTGATGCAGATAACTCGGTGA
ATAAGTTAACCAACAAACTACCTGATCTTTGGCGCAAGATTGAAAGTATC
AACCAACAGCTGTTGCCCTTGGGAAACATCTCTGACAACATGGACAGAAT
ACGAGAACTAATTCAGCAGGCCAGAGATGCTGCCAGTAAGGTTGCTGTCC
CCATGAGGTTCAATGGTAAATCTGGAGTCGAAGTCCGACTGCCAAATGAC
CTGGAAGATTTGAAAGGATATACATCTCTGTCCTTGTTTCTCCAAAGGCC
CAACTCAAGAGAAAATGGGGGTACTGAGAATATGTTTGTGATGTACCTTG
GAAATAAAGATGCCTCCCGGGACTACATCGGCATGGCAGTTGTGGATGGC
CAGCTCACCTGTGTCTACAACCTGGGGGACCGTGAGGCTGAACTCCAAGT
GGACCAGATCTTGACCAAGAGTGAGACTAAGGAGGCAGTTATGGATCGGG
TGAAATTTCAGAGAATTTATCAGTTTGCAAGGCTTAATTACACCAAAGGA
GCCACATCCAGTAAACCAGAAACACCCGGAGTCTATGACATGGATGGTAG
AAATAGCAATACACTCCTTAATTTGGATCCTGAAAATGTTGTATTTTATG
TTGGAGGTTACCCACCTGATTTTAAACTTCCCAGTCGACTAAGTTTCCCT
CCATACAAAGGTTGTATTGAATTAGATGACCTCAATGAAAATGTTCTGAG
CTTGTACAACTTCAAAAAAACATTCAATCTCAACACAACTGAAGTGGAGC
CTTGTAGAAGGAGGAAGGAAGAGTCAGACAAAAATTATTTTGAAGGTACG
GGCTATGCTCGAGTTCCAACTCAACCACATGCTCCCATCCCAACCTTTGG
ACAGACAATTCAGACCACCGTGGATAGAGGCTTGCTGTTCTTTGCAGAAA
ACGGGGATCGCTTCATATCTCTAAATATAGAAGATGGCAAGCTCATGGTG
AGATACAAACTGAATTCAGAGCTACCAAAAGAGAGAGGAGTTGGAGACGC
CATAAACAACGGCAGAGACCATTCGATTCAGATCAAAATTGGAAAACTCC
AAAAGCGTATGTGGATAAATGTGGACGTTCAAAACACTATAATTGATGGT
GAAGTATTTGATTTCAGCACATATTATCTGGGAGGAATTCCAATTGCAAT
CAGGGAAAGATTTAACATTTCTACGCCTGCTTTCCGAGGCTGCATGAAAA
ATTTGAAGAAAACCAGTGGTGTCGTTAGATTGAATGATACTGTGGGAGTA
ACCAAAAAGTGCTCGGAAGACTGGAAGCTTGTGCGATCTGCCTCATTCTC
CAGAGGAGGACAATTGAGTTTCACTGATTTGGGCTTACCACCTACTGACC
ACCTCCAGGCCTCATTTGGATTTCAGACCTTTCAACCCAGTGGCATATTA
TTAGATCATCAGACATGGACAAGGAACCTGCAGGTCACTCTGGAAGATGG
TTACATTGAATTGAGCACCAGCGATAGCGGCGGCCCAATTTTTAAATCTC
CACAGACGTATATGGATGGTTTACTGCATTATGTATCTGTAATAAGCGAC
AACTCTGGACTACGGCTTCTCATCGATGACCAGCTTCTGAGAAATAGCAA
AAGGCTAAAACACATTTCAAGTTCCCGGCAGTCTCTGCGTCTGGGCGGGA
GCAATTTTGAGGGTTGTATTAGCAATGTTTTTGTCCAGAGGTTATCACTG
AGTCCTGAAGTCCTAGATTTGACCAGTAACTCTCTCAAGAGAGATGTGTC
CCTGGGAGGCTGCAGTTTAAACAAACCACCTTTTCTAATGTTGCTTAAAG
GTTCTACCAGGTTTAACAAGACCAAGACTTTTCGTATCAACCAGCTGTTG
CAGGACACACCAGTGGCCTCCCCAAGGAGCGTGAAGGTGTGGCAAGATGC
TTGCTCACCACTTCCCAAGACCCAGGCCAATCATGGAGCCCTCCAGTTTG
GGGACATTCCCACCAGCCACTTGCTATTCAAGCTTCCTCAGGAGCTGCTG
AAACCCAGGTCACAGTTTGCTGTGGACATGCAGACAACATCCTCCAGAGG
ACTGGTGTTTCACACGGGCACTAAGAACTCCTTTATGGCTCTTTATCTTT
CAAAAGGACGTCTGGTCTTTGCACTGGGGACAGATGGGAAAAAATTGAGG
ATCAAAAGCAAGGAGAAATGCAATGATGGGAAATGGCACACGGTGGTGTT
TGGCCATGATGGGGAAAAGGGGCGCTTGGTTGTGGATGGACTGAGGGCCC
GGGAGGGAAGTTTGCCTGGAAACTCCACCATCAGCATCAGAGCGCCAGTT
TACCTGGGATCACCTCCATCAGGGAAACCAAAGAGCCTCCCCACAAACAG
CTTTGTGGGATGCCTGAAGAACTTTCAGCTGGATTCAAAACCCTTGTATA
CCCCTTCTTCAAGCTTCGGGGTGTCTTCCTGCTTGGGTGGTCCTTTGGAG
AAAGGCATTTATTTCTCTGAAGAAGGAGGTCATGTCGTCTTGGCTCACTC
TGTATTGTTGGGGCCAGAATTTAAGCTTGTTTTCAGCATCCGCCCAAGAA
GTCTCACTGGGATCCTAATACACATCGGAAGTCAGCCCGGGAAGCACTTA
TGTGTTTACCTGGAGGCAGGAAAGGTCACGGCCTCTATGGACAGTGGGGC
AGGTGGGACCTCAACGTCGGTCACACCAAAGCAGTCTCTGTGTGATGGAC
AGTGGCACTCGGTGGCAGTCACCATAAAACAACACATCCTGCACCTGGAA
CTGGACACAGACAGTAGCTACACAGCTGGACAGATCCCCTTCCCACCTGC
CAGCACTCAAGAGCCACTACACCTTGGAGGTGCTCCAGCCAATTTGACGA
CACTGAGGATCCCTGTGTGGAAATCATTCTTTGGCTGTCTGAGGAATATT
CATGTCAATCACATCCCTGTCCCTGTCACTGAAGCCTTGGAAGTCCAGGG
GCCTGTCAGTCTGAATGGTTGTCCTGACCAGTAA

Its protein sequence is (3333 aa):

(SEQ ID NO: 8)
MAAAARPRGRALGPVLPPTPLLLLVLRVLPACGATARDPGAAAGLSLHPTYFNLAEAARIWATATCGERG
PGEGRPQPELYCKLVGGPTAPGSGHTIQGQFCDYCNSEDPRKAHPVTNAIDGSERWWQSPPLSSGTQYNR
VNLTLDLGQLFHVAYILIKFANSPRPDLWVLERSVDFGSTYSPWQYFAHSKVDCLKEFGREANMAVTRDD
DVLCVTEYSRIVPLENGEVVVSLINGRPGAKNFTFSHTLREFTKATNIRLRFLRTNTLLGHLISKAQRDP
TVTRRYYYSIKDISIGGQCVCNGHAEVCNINNPEKLFRCECQHHTCGETCDRCCTGYNQRRWRPAAWEQS
HECEACNCHGHASNCYYDPDVERQQASLNTQGIYAGGGVCINCQHNTAGVNCEQCAKGYYRPYGVPVDAP
DGCIPCSCDPEHADGCEQGSGRCHCKPNFHGDNCEKCAIGYYNFPFCLRIP1FPVSTPSSEDPVAGDIKG
CDCNLEGVLPEICDAHGRCLCRPGVEGPRCDTCRSGFYSFPICQACWCSALGSYQMPCSSVTGQCECRPG
VTGQRCDRCLSGAYDFPHCQGSSSACDPAGTINSNLGYCQCKLHVEGPTCSRCKLLYWNLDKENPSGCSE
CKCHKAGTVSGTGECRQGDGDCHCKSHVGGDSCDTCEDGYFALEKSNYFGCQGCQCDIGGALSSMCSGPS
GVCQCREHVVGKVCQRPENNYYFPDLHHMKYEIEDGSTPNGRDLRFGFDPLAFPEFSWRGYAQMTSVQND
VRITLNVGKSSGSLFRVILRYVNPGTEAVSGHITIYPSWGAAQSKEIIFLPSKEPAFVTVPGNGFADPFS
ITPGIWVACIKAEGVLLDYLVLLPRDYYEASVLQLPVTEPCAYAGPPQENCLLYQHLPVTRFPCTLACEA
RHFLLDGEPRPVAVRQPTPAHPVMVDLSGREVELHLRLRIPQVGHYVVVVEYSTEAAQLFVVDVNVKSSG
SVLAGQVNIYSCNYSVLCRSAVIDHMSRIAMYELLADADIQLKGHMARFLLHQVCIIPIEEFSAEYVRPQ
VHCIASYGRFVNQSATCVSLAHETPPTALILDVLSGRPFPHLPQQSSPSVDVLPGVTLKAPQNQVTLRGR
VPHLGRYVFVIHFYQAAHPTFPAQVSVDGGWPRAGSFHASFCPHVLGCRDQVIAEGQIEFDISEPEVAAT
VKVPEGKSLVLVRVLVVPAENYDYQILHKKSMDKSLEFITNCGKNSFYLDPQTASRFCKNSARSLVAFYH
KGALPCECHPTGATGPHCSPEGGQCPCQPNVIGRQCTRCATGHYGFPRCKPCSCGRRLCEEMTGQCRCPP
RTVRPQCEVCETHSFSFHPMAGCEGCNCSRRGTIEAAMPECDRDSGQCRCKPRITGRQCDRCASGFYRFP
ECVPCNCNRDGTEPGVCDPGTGACLCKENVEGTECNVCREGSFHLDPANLKGCTSCFCFGVNNQCHSSHK
RRTKFVDMLGWHLETADRVDIPVSFNPGSNSMVADLQELPATIHSASWVAPTSYLGDKVSSYGGYLTYQA
KSFGLPGDMVLLEKKPDVQLTGQHMSIIYEETNTPRPDRLHHGRVHVVEGNFRHASSRAPVSREELMTVL
SRLADVRIQGLYFTETQRLTLSEVGLEEASDTGSGRIALAVEICACPPAYAGDSCQGCSPGYYRDHKGLY
TGRCVPCNCNGHSNQCQDGSGICVNCQHNTAGEHCERCQEGYYGNAVHGSCRACPCPHTNSFATGCVVNG
GDVRCSCKAGYTGTQCERCAPGYFGNPQKFGGSCQPCSCNSNGQLGSCHPLTGDCINQEPKDSSPAEECD
DCDSCVMTLLNDLATMGEQLRLVKSQLQGLSASAGLLEQMRHMETQAKDLRNQLLNYRSAISNHGSKIEG
LERELTDLNQEFETLQEKAQVNSRKAQTLNNNVNRATQSAKELDVKIKNVIRNVHILLKQISGTDGEGNN
VPSGDFSREWAEAQRMMRELRNRNFGKHLREAEADKRESQLLLNRIRTWQKTHQGENNGLANSIRDSINE
YEAKLSDLRARLQEAAAQAKQANGLNQENERALGAIQRQVKEINSLQSDFTKYLTTADSSLLQTNIALQL
MEKSQKEYEKLAASLNEARQELSDKVRELSRSAGKTSLVEEAEKHARSLQELAKQLEEIKRNASGDELVR
CAVDAATAYENILNAIKAAEDAANRAASASESALQTVIKEDLPRKAKTLSSNSDKLLNEAKMTQKKLKQE
VSPALNNLQQTLNIVTVQKEVIDTNLTTLRDGLHGIQRGDIDAMISSAKSMVRKANDITDEVLDGLNPIQ
TDVERIKDTYGRTQNEDFKKALTDADNSVNKLTNKLPDLWRKIESINQQLLPLGNISDNMDRIRELIQQA
RDAASKVAVPMRFNGKSGVEVRLPNDLEDLKGYTSLSLFLQRPNSRENGGTENMFVMYLGNKDASRDYIG
MAVVDGQLTCVYNLGDREAELQVDQILTKSETKEAVMDRVKFQRIYQFARLNYTKGATSSKPETPGVYDM
DGRNSNTLLNLDPENVVFYVGGYPPDFKLPSRLSFPPYKGCIELDDLNENVLSLYNFKKTFNLNTTEVEP
CRRRKEESDKNYFEGTGYARVPTQPHAPIPTFGQTIQTTVDRGLLFFAENGDRFISLNIEDGKLMVRYKL
NSELPKERGVGDAINNGRDHSIQIKIGKLQKRMWINVDVQNTIIDGEVFDFSTYYLGGIPIAIRERFNIS
TPAFRGCMKNLKKTSGVVRLNDTVGVTKKCSEDWKLVRSASFSRGGQLSFTDLGLPPTDHLQASFGFQTF
QPSGILLDHQTWTRNLQVTLEDGYIELSTSDSGGPIFKSPQTYMDGLLHYVSVISDNSGLRLLIDDQLLR
NSKRLKHISSSRQSLRLGGSNFEGCISNVFVQRLSLSPEVLDLTSNSLKRDVSLGGCSLNKPPFLMLLKG
STRFNKTKTFRINQLLQDTPVASPRSVKVWQDACSPLPKTQANHGALQFGDIPTSHLLFKLPQELLKPRS
QFAVDMQTTSSRGLVFHTGTKNSFMALYLSKGRLVFALGTDGKKLRIKSKEKCNDGKWHTVVFGHDGEKG
RLVVDGLRAREGSLPGNSTISIRAPVYLGSPPSGKPKSLPTNSFVGCLKNFQLDSKPLYTPSSSFGVSSC
LGGPLEKGIYFSEEGGHVVLAHSVLLGPEFKLVFSIRPRSLTGILIHIGSQPGKHLCVYLEAGKVTASMD
SGAGGTSTSVTPKQSLCDGQWHSVAVTIKQHILHLELDTDSSYTAGQIPFPASTQEPLHLGGAPANLTT
IRIPVWKSFFGCLRNIHVNHIPVPVTEALEVQGPVSLNGCPDQ

Preferably, the LAMC2 (γ2 chain of laminin 5) is characterized by the sequence disclosed in the NCBI Data Bank with the Accession no.: NM_018891. Its cDNA sequence is:

(SEQ ID NO: 9)
ATGCCTGCGCTCTGGCTGGGCTGCTGCCTCTGCTTCTCGCTCCTCCTGCC
CGCAGCCCGGGCCACCTCCAGGAGGGAAGTCTGTGATTGCAATGGGAAGT
CCAGGCAGTGTATCTTTGATCGGGAACTTCACAGACAAACTGGTAATGGA
TTCCGCTGCCTCAACTGCAATGACAACACTGATGGCATTCACTGCGAGAA
GTGCAAGAATGGCTTTTACCGGCACAGAGAAAGGGACCGCTGTTTGCCCT
GCAATTGTAACTCCAAAGGTTCTCTTAGTGCTCGATGTGACAACTCCGGA
CGGTGCAGCTGTAAACCAGGTGTGACAGGAGCCAGATGCGACCGATGTCT
GCCAGGCTTCCACATGCTCACGGATGCGGGGTGCACCCAAGACCAGAGAC
TGCTAGACTCCAAGTGTGACTGTGACCCAGCTGGCATCGCAGGGCCCTGT
GACGCGGGCCGCTGTGTCTGCAAGCCAGCTGTCACTGGAGAACGCTGTGA
TAGGTGTCGATCAGGTTACTATAATCTGGATGGGGGGAACCCTGAGGGCT
GTACCCAGTGTTTCTGCTATGGGCATTCAGCCAGCTGCCGCAGCTCTGCA
GAATACAGTGTCCATAAGATCACCTCTACCTTTCATCAAGATGTTGATGG
CTGGAAGGCTGTCCAACGAAATGGGTCTCCTGCAAAGCTCCAATGGTCAC
AGCGCCATCAAGATGTGTTTAGCTCAGCCCAACGACTAGACCCTGTCTAT
TTTGTGGCTCCTGCCAAATTTCTTGGGAATCAACAGGTGAGCTATGGTCA
AAGCCTGTCCTTTGACTACCGTGTGGACAGAGGAGGCAGACACCCATCTG
CCCATGATGTGATTCTGGAAGGTGCTGGTCTACGGATCACAGCTCCCTTG
ATGCCACTTGGCAAGACACTGCCTTGTGGGCTCACCAAGACTTACACATT
CAGGTTAAATGAGCATCCAAGCAATAATTGGAGCCCCCAGCTGAGTTACT
TTGAGTATCGAAGGTTACTGCGGAATCTCACAGCCCTCCGCATCCGAGCT
ACATATGGAGAATACAGTACTGGGTACATTGACAATGTGACCCTGATTTC
AGCCCGCCCTGTCTCTGGAGCCCCAGCACCCTGGGTTGAACAGTGTATAT
GTCCTGTTGGGTACAAGGGGCAATTCTGCCAGGATTGTGCTTCTGGCTAC
AAGAGAGATTCAGCGAGACTGGGGCCTTTTGGCACCTGTATTCCTTGTAA
CTGTCAAGGGGGAGGGGCCTGTGATCCAGACACAGGAGATTGTTATTCAG
GGGATGAGAATCCTGACATTGAGTGTGCTGACTGCCCAATTGGTTTCTAC
AACGATCCGCACGACCCCCGCAGCTGCAAGCCATGTCCCTGTCATAACGG
GTTCAGCTGCTCAGTGATGCCGGAGACGGAGGAGGTGGTGTGCAATAACT
GCCCTCCCGGGGTCACCGGTGCCCGCTGTGAGCTCTGTGCTGATGGCTAC
TTTGGGGACCCCTTTGGTGAACATGGCCCAGTGAGGCCTTGTCAGCCCTG
TCAATGCAACAACAATGTGGACCCCAGTGCCTCTGGGAATTGTGACCGGC
TGACAGGCAGGTGTTTGAAGTGTATCCACAACACAGCCGGCATCTACTGC
GACCAGTGCAAAGCAGGCTACTTCGGGGACCCATTGGCTCCCAACCCAGC
AGACAAGTGTCGAGCTTGCAACTGTAACCCCATGGGCTCAGAGCCTGTAG
GATGTCGAAGTGATGGCACCTGTGTTTGCAAGCCAGGATTTGGTGGCCCC
AACTGTGAGCATGGAGCATTCAGCTGTCCAGCTTGCTATAATCAAGTGAA
GATTCAGATGGATCAGTTTATGCAGCAGCTTCAGAGAATGGAGGCCCTGA
TTTCAAAGGCTCAGGGTGGTGATGGAGTAGTACCTGATACAGAGCTGGAA
GGCAGGATGCAGCAGGCTGAGCAGGCCCTTCAGGACATTCTGAGAGATGC
CCAGATTTCAGAAGGTGCTAGCAGATCCCTTGGTCTCCAGTTGGCCAAGG
TGAGGAGCCAAGAGAACAGCTACCAGAGCCGCCTGGATGACCTCAAGATG
ACTGTGGAAAGAGTTCGGGCTCTGGGAAGTCAGTACCAGAACCGAGTTCG
GGATACTCACAGGCTCATCACTCAGATGCAGCTGAGCCTGGCAGAAAGTG
AAGCTTCCTTGGGAAACACTAACATTCCTGCCTCAGACCACTACGTGGGG
CCAAATGGCTTTAAAAGTCTGGCTCAGGAGGCCACAAGATTAGCAGAAAG
CCACGTTGAGTCAGCCAGTAACATGGAGCAACTGACAAGGGAAACTGAGG
ACTATTCCAAACAAGCCCTCTCACTGGTGCGCAAGGCCCTGCATGAAGGA
GTCGGAAGCGGAAGCGGTAGCCCGGACGGTGCTGTGGTGCAAGGGCTTGT
GGAAAAATTGGAGAAAACCAAGTCCCTGGCCCAGCAGTTGACAAGGGAGG
CCACTCAAGCGGAAATTGAAGCAGATAGGTCTTATCAGCACAGTCTCCGC
CTCCTGGATTCAGTGTCTCGGCTTCAGGGAGTCAGTGATCAGTCCTTTCA
GGTGGAAGAAGCAAAGAGGATCAAACAAAAAGCGGATTCACTCTCAAGCC
TGGTAACCAGGCATATGGATGAGTTCAAGCGTACACAGAAGAATCTGGGA
AACTGGAAAGAAGAAGCACAGCAGCTCTTACAGAATGGAAAAAGTGGGAG
AGAGAAATCAGATCAGCTGCTTTCCCGTGCCAATCTTGCTAAAAGCAGAG
CACAAGAAGCACTGAGTATGGGCAATGCCACTTTTTATGAAGTTGAGAGC
ATCCTTAAAAACCTCAGAGAGTTTGACCTGCAGGTGGACAACAGAAAAGC
AGAAGCTGAAGAAGCCATGAAGAGACTCTCCTACATCAGCCAGAAGGTTT
CAGATGCCAGTGACAAGACCCAGCAAGCAGAAAGAGCCCTGGGGAGCGCT
GCTGCTGATGCACAGAGGGCAAAGAATGGGGCCGGGGAGGCCCTGGAAAT
CTCCAGTGAGATTGAACAGGAGATTGGGAGTCTGAACTTGGAAGCCAATG
TGACAGCAGATGGAGCCTTGGCCATGGAAAAGGGACTGGCCTCTCTGAAG
AGTGAGATGAGGGAAGTGGAAGGAGAGCTGGPAAGGAAGGAGCTGGAGTT
TGACACGAATATGGATGCAGTACAGATGGTGATTACAGAAGCCCAGAAGG
TTGATACCAGAGCCAAGAACGCTGGGGTTACAATCCAAGACACACTCAAC
ACATTAGACGGCCTCCTGCATCTGATGGGTATGTGA

Its protein sequence is:

(SEQ ID NO: 10)
MPALWLGCCLCFSLLLPAARATSRREVCDCNGKSRQCIFDRELHRQTGNG
FRCLNCNDNTDGIHCEKCKNGFYRHRERDRCLPCNCNSKGSLSARCDNSG
RCSCKPGVTGARCDRCLPGFHMLTDAGCTQDQRLLDSKCDCDPAGIAGPC
DAGRCVCKPAVTGERCDRCRSGYYNLDGGNPEGCTQCFCYGHSASCRSSA
EYSVHKITSTFHQDVDGWKAVQRNGSPAKLQWSQRHQDVFSSAQRLDPVY
FVAPAKFLGNQQVSYGQSLSFDYRVDRGGRHPSAHDVILEGAGLRITAPL
MPLGKTLPCGLTKTYTFRLNEHPSNNWSPQLSYFEYRRLLRNLTALRIRA
TYGEYSTGYIDNVTLISARPVSGAPAPWVEQCICPVGYKGQFCQDCASGY
KRDSARLGPFGTCIPCNCQGGGACDPDTGDCYSGDENPDIECADCPIGFY
NDPHDPRSCKPCPCHNGFSCSVMPETEEVVCNNCPPGVTGARCELCADGY
FGDPFGEHGPVRPCQPCQCNNNVDPSASGNCDRLTGRCLKCIHNTAGIYC
DQCKAGYFGDPLAPNPADKCRACNCNPMGSEPVGCRSDGTCVCKPGFGGP
NCEHGAFSCPACYNQVKIQMDQFMQQLQRMEALISKAQGGDGVVPDTELE
GRMQQAEQALQDILRDAQISEGASRSLGLQLAKVRSQENSYQSRLDDLKM
TVERVRALGSQYQNRVRDTHRLITQMQLSLAESEASLGNTNIPASDHYVG
PNGFKSLAQEATRLAESHVESASNMEQLTRETEDYSKQALSLVRKALHEG
VGSGSGSPDGAVVQGLVEKLEKTKSLAQQLTREATQAEIEADRSYQHSLR
LLDSVSRLQGVSDQSFQVEEAKRIKQKADSLSSLVTRHMDEFKRTQKNLG
NWKEEAQQLLQNGKSGREKSDQLLSRANLAKSRAQEALSMGNATFYEVES
ILKNLREFDLQVDNRKAEAEEAMKRLSYISQKVSDASDKTQQAERALGSA
AADAQRAKNGAGEALEISSEIEQEIGSLNLEANVTADGALAMEKGLASLK
SEMREVEGELERKELEFDTNMDAVQMVITEAQKVDTRAKNAGVTIQDTLN
TLDGLLHLMGM

Included in the present invention are also nucleic acid sequences derived from the sequences shown below, e.g. functional fragments, mutants, derivatives, analogues, and sequences having a % of identity of at least 70% with the below sequences.

In the context of the present invention, the cDNA, the gene, the mRNA, the polynucleotide or the protein encoded therefrom herein mentioned comprise also their functional fragments, functional analogous, derivatives, variants, isoforms, orthologues or homologous, splicing variants, functional mutants, etc.

The term gene (or cDNA) herein also includes corresponding orthologous or homologous genes, isoforms, variants, allelic variants, functional derivatives, functional fragments thereof. The expression “protein” is intended to include also the corresponding protein encoded from a corresponding orthologous or homologous genes, functional mutants, functional derivatives, functional fragments or analogues, isoforms thereof.

In the context of the present invention, the term “polypeptide” or “protein” includes:
i. the whole protein, allelic variants and orthologs thereof;
ii. any synthetic, recombinant or proteolytic functional fragment;
iii. any functional equivalent, such as, for example, synthetic or recombinant functional analogues.

In the present invention “functional mutants” of the protein are mutants that may be generated by mutating one or more amino acids in their sequences and that maintain their activity. Indeed, in the present invention the protein encoded by the transgene, if required, can be modified in vitro and/or in vivo, for example by glycosylation, myristoylation, amidation, carboxylation or phosphorylation, and may be obtained, for example, by synthetic or recombinant techniques known in the art. The term “derivative” as used herein in relation to a protein means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. As used herein, the term “derivatives” also refers to longer or shorter polypeptides having e.g. a percentage of identity of at least 41% , preferably at least 41.5%, 50%, 54.9% , 60%, 61.2%, 64.1%, 65%, 70% or 75%, more preferably of at least 85%, as an example of at least 90%, and even more preferably of at least 95% with the herein disclosed genes and sequences, or with an amino acid sequence of the correspondent region encoded from orthologous or homologous gene thereof. The term “analogue” as used herein referring to a protein means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A “derivative” may be a nucleic acid molecule, as a DNA molecule, coding the polynucleotide as above defined, or a nucleic acid molecule comprising the polynucleotide as above defined, or a polynucleotide of complementary sequence. In the context of the present invention the term “derivatives” also refers to longer or shorter polynucleotides and/or polynucleotides having e.g. a percentage of identity of at least 41% , 50%, 60%, 65%, 70% or 75%, more preferably of at least 85%, as an example of at least 90%, and even more preferably of at least 95% or 100% with the sequences herein discloses or with their complementary sequence or with their DNA or RNA corresponding sequence. The term “derivatives” and the term “polynucleotide” also include modified synthetic oligonucleotides. The modified synthetic oligonucleotide are preferably LNA (Locked Nucleic Acid), phosphoro-thiolated oligos or methylated oligos, morpholinos, 2′-O-methyl, 2′-O-methoxyethyl oligonucleotides and cholesterol-conjugated 2′-O-methyl modified oligonucleotides (antagomirs). The term “derivative” may also include nucleotide analogues, i.e. a naturally occurring ribonucleotide or deoxyribonucleotide substituted by a non-naturally occurring nucleotide. The term “derivatives” also includes nucleic acids or polypeptides that may be generated by mutating one or more nucleotide or amino acid in their sequences, equivalents or precursor sequences. The term “derivatives” also includes at least one functional fragment of the polynucleotide. In the context of the present invention “functional” is intended for example as “maintaining their activity”. As used herein “fragments” refers to polynucleotides having preferably a length of at least 1000 nucleotides, 1100 nucleotide, 1200 nucleotides, 1300 nucleotides, 1400 nucleotides, 1500 nucleotides or to polypeptide having preferably a length of at least 50 aa, 100 aa, 150 aa, 200 aa, 250 aa, 300 aa, . . . . The term “polynucleotide” also refers to modified polynucleotides.

The term “functional fragment” or “functional derivative” may be understood as maintaining the same activity of the protein. “Derivatives” may be recombinant or synthetic. The term “derivative” as used herein in relation to a protein means a chemically modified protein or an analogue thereof, wherein at least one substituent is not present in the unmodified protein or an analogue thereof, i.e. a protein which has been covalently modified. Typical modifications are amides, carbohydrates, alkyl groups, acyl groups, esters and the like

In the context of the present invention, the stratified epithelia above described is preferably epidermis, cornea, oral mucosa, etc.

Fibrin guarantees a solid-biological substrate to the cells allowing their growth in order to obtain a flap of genetically modified cells adhered to said substrate.

Fibrin is a poorly soluble fraction produced by the specific hydrolysis carried out by the thrombin of the fibrinogen alpha A chain and B beta chain to release fibrinopeptides A and B.

Thrombin is a protease that can act on fibrinogen to produce fibrin. In the composition of the present invention, thrombin may be present in a catalytically effective amount to convert fibrinogen into fibrin. Fibrinogen and thrombin are preferably derived from humans but may also be derived from other animals such as monkey, pig, rat, dog, bovine, etc.

Fibrinogen and thrombin for use in the present invention may be commercially available products.

Preferably, the fibrinogen and thrombin composition of the present invention also includes calcium chloride (which may be in hydrate form), aprotinin, sodium chloride.

An example of the composition of the present invention (for 12 ml total, i.e. 6 ml of fibrinogen mixed with 6 ml of thrombin) consists of:

Fibrinogen from 20 to 100 mg/ml, preferably from 20 to 50 mg/ml, more preferably from 20 to 40 mg/ml, even more preferably from 20 to 25 mg/ml;

Thrombin from 1 to 10 IU/ml, preferably from 3 to 8 IU/ml, more preferably from 2 to 4 IU/ml;

Aprotinin 1100 IU/ml to 2000 IU/ml;

Buffer consisting of NaCl (1-11%) and CaCl2 (1-1.5 mM).

Preferably, fibrin gels consist of fibrinogen (23.1 mg/ml) and thrombin (3.1 IU/ml) in NaCl (1%), CaCl₂ (1 mM) and Aprotinin (1,786 KIU/ml).

In a preferred form of the invention, the physiological solution (NaCl 0.9%) is used in the preparation of aprotinin. 10% NaCl is preferably used in the buffer to dissolve fibrinogen and thrombin.

Aprotinin and/or sodium chloride, etc., may be added to the fibrinogen before mixing the composition with the thrombin.

Sodium chloride can be added to the thrombin before mixing the composition with fibrinogen. Before releasing the fibrin gels, they are subjected to conformity controls as per Table 3.

TABLE 3
Features for fibrin gel releasing

| Parameters | Features |
|---|---|
| Transparence | Opacity absence |
| Uniformity | Uniform fibrin gel |
| Structural integrity | Hole absence |

The fibrin composition (including fibrinogen and thrombin) as described above may be used to coat a surface of the support for the preparation of cell flaps. The support may be of any type known to the art expert, provided that the cells can be cultivated on it. Support examples include untreated petri dish plates for cell cultures. Other support examples are culture plates or plates having 6 to 96 wells characterized by being able to facilitate fibrin detachment. Non-limiting examples of support material are: glass, modified glass, polystyrene, ceramic, polymethacrylate and cell culture plates, provided that the material is capable of promoting fibrin detachment.

The above-described composition comprising fibrinogen and thrombin can be applied to a surface of the support and left at room temperature for 10-15 minutes or until complete polymerization. The support thus obtained can be stored under sterile conditions at 4° C.

The term “confluence” in the context of the present invention indicates preferably the state in which the cells have such a density that there is no space among them and can be evaluated by the microscope.

The term “subconfluence” in the context of the present invention indicates preferably the state in which the optionally genetically modified cells, e.g. epithelial cells, have such density that are still partially surrounded by feeder cells, that state may be evaluated by the microscope.

Examples of genetically modified cells that can be cultivated include, but are not limited to, cardiac cells, skeletal cells, mature skeletal muscle cells, smooth muscle cells, corneal epithelial cells, epithelial cells of the oral mucosa and epidermal cells. Preferably, said cells are corneal epithelial cells, epithelial cells of the oral mucous and epidermal cells, more preferably dermal cells. The cells can be derived from humans or animals. Cells can be genetically modified and then cultured directly from the source, like an animal, or can be cultured cells of a cell line stabilized or not.

Cell culture can be carried out by any method or under any condition provided that the culture is conducted on the surface of the fibrin-coated support.

Once subconfluence is reached, the culture medium is removed and the resulting cellular flap can be washed and detached from the support using, for example, pliers.

Any method known to the art expert for genetic modification of cells can be used in the present invention. In a preferred aspect of the invention, the genetically modified cells described herein are characterized by the fact that exogenous nucleic acid has been introduced by the use of a viral vector, for example in the form of a viral expression construct, more preferably a Retroviral vector. Alternatively, the genetically modified cells described herein are characterized by the fact that exogenous nucleic acid is or comprises a construct of non-viral expression.

Preferably, in the vector as described above, the polynucleotide (or exogenous nucleic acid) is under the control of a promoter capable of expressing said polynucleotide efficiently.

The polynucleotide sequence in the vector is operatively linked to an appropriate expression control sequence (promoter) to direct the synthesis of the mRNA. Examples of promoters include the immediate promoter of early cytomegalovirus (CMV) genes, thymidine kinase HSV, early and late SV40, LTRs from retrovirus, preferably derived from murine leukemia virus (MLV). The vectors may also contain one or more selectable gene markers.

As used herein, the term “genetically modified cell” refers to a host cell that has been transduced, transformed or transfected with the polynucleotide or with the vector as described above.

As examples of suitable host cells, bacterial cells, fungal and yeast cells, insect cells, plant cells, animal cells, preferably human cells, and more preferably cells from biopsies of the skin, can be cited.

The introduction of the polynucleotide or vector previously described in the host cell may be carried out using methods known to the art expert, such as calcium phosphate transfection, DEAE-dextran mediated transfection, electroporation, lipofection, microinjection, viral infection, thermal shock, cell fusion, . . . .

The invention will be now illustrated by means of non-limiting examples referring to the following figures.

FIG. 1. Representative pictures of cultured keratinocytes grown on plastic. The image on the right is representative of the flap prior to detachment and assembly for transport.

FIG. 2. Representative images of the flap detachment with Dispase II and two preparations of the flap originated from plastic. The center image shows a flap not conforming to the release due to the presence of air bubbles, while the photo on the right represents the image of a flap conforming to the release.

FIG. 3. Representative images of the confluences reached by growing keratinocytes on fibrin supports at the time of detachment and preparation for transport.

FIG. 4. Representative images of the preparation of the genetically modified epidermis flap.

### EXAMPLES

Materials and Methods

Isolation of Epidermal Keratinocytes from Biopsy of Human Skin

Primary human keratinocytes are isolated from 2-9 cm² skin biopsies after submission and adhesion to informed consent. The biopsy is subjected to enzymatic digestion in Trypsin-EDTA solution at 37° C. To obtain maximum yield and minimize the risk of toxicity from exposure to trypsin-EDTA or prolonged suspension time of keratinocytes during extraction, till 6 sequential trypsinizations of 30′ each are performed. After each trypsinization the cellular material is recovered and the trypsinizations 1-3 and 4-6 are combined and plated in plastic supports according to the cell yield at a density of 1.33×10⁴ cells/cm² on a feeder layer of lethal irradiated murine cells 3T3-J2 (Rheinwald, J. et al. 1975). The medium used consists of a mixture of Dulbecco's modified Eagle (DMEM) and Ham F12 (2:1) supplemented with 10% fetal bovine serum, 0.5% penicillin-streptomycin, 2% glutamine, insulin (5 μg/ml), adenine (0.18 mM), hydrocortisone (0.4 μg/ml), cholera toxin (0.1 nM) and triiodothyronine (2 nM). After 3 days, this culture medium is removed and replaced with KC (KNO medium containing 10 ng/ml of EGF) which is changed every other day until the subconfluence stage is reached.

Primary Culture and Transduction of Keratinocytes.

After reaching the subconfluence (80-95%), the cells are subjected to enzymatic digestion with trypsin-EDTA for 15′ at 37° C. The cell suspension thus obtained is plated to a density of 1.33×10⁴ cells/cm² on the feeder layer composed of 3T3J2 and AM12 cells (packaging cells) (Mavilio et al., Nature, 2006) producing the retroviral vector carrying the beta3 chain of laminin 5 (8×10⁴ cells/cm²) in a 1:2 ratio. After 3 days, the cells are transferred onto a new 3T3-J2 layer feeder, which is plated on a support of the same size and grown to subconfluence. Once the transduced cells have reached subconfluence, they are trypsinized as previously described. The cells thus obtained are divided into appropriate freezing vials of 1-2×10⁶ cells. In parallel, process control tests are performed. Process controls consist of:

1. Evaluation of the number of transduced cells. A number from 1000 to 10,000 cells is plated on feeder layer in “multiwells/chambers” for the evaluation of the number of positive cells at the transgene.

2. Evaluation of the “colony forming efficiency” CFE. A number from 500 to 2000 cells is plated on a 100 mm dish and stained after 12 days in rhodamine.

3. Evaluation of the number of 3T3J2 and AM12 cells present in the cell suspension.

Gel Fibrin Production in 144cm² Supports

Fibrin is produced by the inventor and comprises two fibrinogen reagents (23.1 mg/ml) and thrombin (3.1 IU/ml) produced by Kedrion and sold with the commercial name Kolfib. The production process involves three phases:

1. Preparation of fibrinogen solution and thrombin

2. Preparation of fibrin support

3. Fibrin compliance test

1. A thrombin (kedrion) vial containing 625 IU or 1250 IU of thrombin is reconstituted in 10 ml of buffer consisting of NaCl (1.1%) and CaCl2 (1 mM). The entire content is then transferred to a 50 ML tube to which other 10 ML buffer will be added. If the starting vial contained 625 UI of thrombin, a 1:5 dilution of the reconstituted solution is made, e.g. 5 ML of the reconstituted solution are transferred in a new 50 ml tube and after 20 ML of buffer are added. If the starting vial contained 1250 UI of thrombin, a dilution of 1:10 of the reconstituted solution is made, e.g 18 ML of the reconstituted solution are transferred in a new 50 ML tube and other 162 ml of buffer are added. The solution is prepared at room temperature and examined to ensure that there are no solubilized thrombin solutions. A 120 mg or 240 mg fibrinogen vial is solubilized in 2.59 ML or 5.184 ML of buffer containing NaCl (1.73%) and CaCl2 (1.23 mM) and aprotinin (3574 KIU/ml). The reconstituted solution is incubated at 36.5° C. for 30 to 60 minutes to complete the solubilization.

2. The fibrin gel (or fibrin substrate) is prepared in a 144 cm² support in untreated plates for cell culture. To obtain a 100 mm thick gel, 6 ML of thrombin solution and 6 ML of fibrinogen solution are mixed to obtain a homogeneous mixture. The plates thus prepared are left at room temperature for 10-15 min until full polymerization and then stored at 4° C. for up to one month.

3. Before releasing the fibrin gel are subjected to compliance checks (see Table 3).

Flaps Preparation

The first step in the preparation of the genetically modified epidermis flap consists in plating 3T3-J2 feeder cells on the 144 cm² fibrin support. The feeder cells are plated 2 to 24 h before the keratinocyte plating or at the same time of the thawing of transduced keratinocytes.

The feeder cells are plated on the upper surface of the fibrin, the homogeneous plating of the feeder is controlled through an optical microscope inspection with 10× and 20× magnification. A vial of transduced cells is thawed and plated on supports previously described at a density of 500,000-3,000,000 cells transduced for a surface of 144 cm². The prepared culture is transferred to an incubator at 37° C., 6% CO2 and 99% humidity, monitored and the medium is changed every two days until reaching the subconfluence (7-14 days).

Preparation of Flaps for Transport

Once the subconfluence is reached, the culture medium is removed. The genetically modified epidermis flap is washed three times with a washing and transport solution consisting of DMEM and L-Glutamine. Once the washings have been carried out, the flap is detached from the plastic container by means of sterile forceps and then transferred to the transport container for the flap to which the transport medium will be added. The container is then sealed to keep the environment sterile inside it. At this stage it will be necessary to avoid the formation of air bubbles. The fibrin flap thus prepared has a stability of 36 hours.

### REFERENCES

Barrandon Y, Green H. 1987. Three clonal types of keratinocyte with different capacities for multiplication. Proc Natl Acad Sci USA 84:2302-2306.

Bauer J W, Koller J, Murauer E M, De Rosa L, Enzo E, Carulli S, Bondanza S, Recchia A, Muss W, Diem A, Mayr E, Schlager P, Gratz I K, Pellegrini G, De Luca M. 2017. Closure of a Large Chronic Wound through Transplantation of Gene-Corrected Epidermal Stem Cells. J Invest Dermatol 137:778-781.

Cuono C, Langdon R, McGuire J. 1986. Use of cultured epidermal autografts and dermal allografts as skin replacement after burn injury. Lancet 1:1123-1124.

De Luca M, Albanese E, Bondanza S, Megna M, Ugozzoli L, Molina F, Cancedda R, Santi P L, Bormioli M, Stella M, et al. 1989. Multicentre experience in the treatment of burns with autologous and allogenic cultured epithelium, fresh or preserved in a frozen state. Burns 15:303-309.

De Luca M, Pellegrini G, Green H. 2006. Regeneration of squamous epithelia from stem cells of cultured grafts. Regen Med 1:45-57.

Fine J D. 2010. Inherited epidermolysis bullosa: recent basic and clinical advances. Curr Opin Pediatr 22:453-458.

Gallico G G, 3rd, O'Connor N E, Compton C C, Kehinde O, Green H. 1984. Permanent coverage of large burn wounds with autologous cultured human epithelium. N Engl J Med 311:448-451.

Mavilio F, Pellegrini G, Ferrari S, Di Nunzio F, Di Iorio E, Recchia A, Maruggi G, Ferrari G, Provasi E, Bonini C, Capurro S, Conti A, Magnoni C, Giannetti A, De Luca M. 2006. Correction of junctional epidermolysis bullosa by transplantation of genetically modified epidermal stem cells. Nat Med 12:1397-1402.

Nicholase. O'Connor J M, Susan Banks-Schlegel, Olaniyi Kehinde, Howard Green. . 1981. Grafting of burns with cultured epithelium prepared from autologous epidermal cells. Lancet 1:75-78.

Pellegrini G, Rama P, Matuska S, Lambiase A, Bonini S, Pocobelli A, Colabelli R G, Spadea L, Fasciani R, Balestrazzi E, Vinciguerra P, Rosetta P, Tortori A, Nardi M, Gabbriellini G, Traverso C E, Macaluso C, Losi L, Percesepe A, Venturi B, Corradini F, Panaras A, Di Rocco A, Guatelli P, De Luca M. 2013. Biological parameters determining the clinical outcome of autologous cultures of limbal stem cells. Regen Med 8:553-567.

Pellegrini G, Ranno R, Stracuzzi G, Bondanza S, Guerra L, Zambruno G, Micali G, De Luca M. 1999. The control of epidermal stem cells (holoclones) in the treatment of massive full-thickness burns with autologous keratinocytes cultured on fibrin. Transplantation 68:868-879.

Rama P, Matuska S, Paganoni G, Spinelli A, De Luca M, Pellegrini G. 2010. Limbal stem-cell therapy and long-term corneal regeneration. N Engl J Med 363:147-155.

Rheinwald J G, Green H. 1975. Serial cultivation of strains of human epidermal keratinocytes: the formation of keratinizing colonies from single cells. Cell 6:331-343.

Watt F M, Jordan P W, O'Neill C H. 1988. Cell shape controls terminal differentiation of human epidermal keratinocytes. Proc Natl Acad Sci USA 85:5576-5580.

1. An in vitro method for producing a flap of genetically modified cells on a fibrin substrate, comprising:
a) plating feeder cells on the upper surface of a fibrin substrate so as to obtain a fibrin substrate on which said feeder cells are adhered;
b) plating and cultivating to subconfluence said genetically modified cells on said fibrin substrate onto which feeder cells are adhered, said fibrin substrate being positioned on a solid support so that the genetically modified cells do not interact with the surface of said support so as to obtain a flap of genetically modified cells adhered to said fibrin substrate; and
c) detaching the flap of genetically modified cells adhered to said fibrin substrate from the support in a form similar to a sheet to obtain a flap of genetically modified cells on said fibrin substrate.

2. The method according to claim 1, wherein the feeder cells are plated on the fibrin substrate from 2 to 24 hours before plating the genetically modified cells.

3. The method according to claim 1, further comprising:
before step c), the steps:
b′) removing the culture medium and/or
b″) washing the flap of genetically modified cells adhered to said fibrin substrate with a washing solution
and/or after step c), the step of:
d) placing the obtained flap of genetically modified cells on fibrin substrate in a transport container.

4. The method according to claim 1, wherein the fibrin substrate has dimensions of from about 0.32 cm² to about 300 cm².

5. The method according to claim 1, wherein the fibrin substrate comprises from about 20 to about 100 mg/ml of fibrinogen and from about 1 to about 10 IU/ml of thrombin.

6. The method according to claim 5, wherein the fibrin substrate comprises from about 20 to about 50 mg/ml of fibrinogen, and from about 3 to about 8 IU/ml of thrombin.

7. The method according to claim 6, wherein the fibrin substrate comprises from about 20 to about 25 mg/ml of fibrinogen and from about 2 to about 4 IU/ml of thrombin.

8. The method according to claim 7, wherein the fibrin substrate comprises about 23.1 mg/ml of fibrinogen and about 3.1 IU/ml of thrombin.

9. The method according to claim 1, wherein said genetically modified cells are epithelial cells.

10. The method according to claim 9 wherein said genetically modified cells are epidermal cells.

11. The method according to claim 9 wherein said genetically modified cells are keratinocytes.

12. The method according to claim 1, wherein said genetically modified cells have been transduced with a gene or a cDNA selected from the group consisting of:
a) at least one chain selected from the group consisting of: beta-3, α3 and γ2 chain of laminin-5, and/or
b) collagen 17 and/or
c) at least one α6β4 integrin and/or
d) collagen 7 and/or
e) keratin 5 and Keratin 14 and/or
f) Plectin.

13. The method according to claim 1, wherein said genetically modified cells have been transduced with a gene or a cDNA selected from the group consisting of: beta-3 chain of laminin 5, collagen 7 and collagen 17.

14. A flap of genetically modified cells on a fibrin substrate, obtainable by the method of claim 1.

15. A flap of genetically modified cells on a fibrin substrate, wherein said genetically modified cells are epithelial cells.

16. The flap of genetically modified cells on a fibrin substrate according to claim 15 wherein said genetically modified cells are epidermal cells.

17. The flap of genetically modified cells on a fibrin substrate according to claim 16 wherein said cells are keratinocytes.

18. The flap according to claim 14 wherein the genetically modified cells are transduced with a gene or a cDNA selected from the group consisting of:
a) at least one chain selected from the group consisting of: beta-3, α3 and γ2 chain of laminin-5, and/or
b) collagen 17 and/or
c) at least one α6β4 integrin and/or
d) collagen 7 and/or
e) keratin 5 and Keratin 14 and/or
f) Plectin.

19. The flap according to claim 14 wherein the genetically modified cells are transduced with a gene or a cDNA selected from the group consisting of: beta-3 chain of laminin 5, collagen 7 and collagen 17.

20. The flap according to claim 14 wherein the genetically modified cells are cells that have been transduced with a retroviral vector.

21. The flap according to claim 14 wherein the fibrin substrate comprises from about 20 to about 100 mg/ml of fibrinogen and from about 1 to about 10 IU/ml of thrombin.

22. The flap according to claim 21 wherein the fibrin substrate comprises from about 20 to about 50 mg/ml of fibrinogen, and from about 3 to about 8 IU/ml of thrombin.

23. The flap according to claim 22 wherein the fibrin substrate comprises from about 20 to about 25 mg/ml of fibrinogen and from about 2 to about 4 IU/ml of thrombin.

24. The flap according to claim 23 wherein the fibrin substrate comprises about 23.1 mg/ml of fibrinogen and about 3.1 IU/ml of thrombin.

25-29. (canceled)
